(19) United States
(12) Patent Application Publication
(10) Pub. No.: US 2007/0021421 A1
(43) Pub. Date: Jan. 25, 2007

(54) Measurement of gait dynamics and use of beta-blockers to detect, prognose, prevent and treat amyotrophic lateral sclerosis

(76) Inventor: Thomas G. Hampton, Framingham, MA (US)

Correspondence Address: LAHIVE & COCKFIELD, LLP, ONE POST OFFICE SQUARE, BOSTON, MA, 02109-2127, US

(21) Appl. No.: 11/492,444

(22) Filed: Jul. 25, 2006

Related U.S. Application Data
(60) Provisional application No. 60/702,377, filed on Jul. 25, 2005.
(60) Provisional application No. 60/735,389, filed on Nov. 11, 2005.

(51) Int. Cl. A61K 31/5377; A61K 31/5375; A61K 31/403; A61K 31/138

(52) U.S. Cl. 514/237.5; 514/411; 514/651

(57) ABSTRACT

The present invention, at least in part, provides methods of improved early diagnosis of neurodegenerative disease, e.g., ALS, in a subject via measurement of the gait dynamics of the subject (e.g., via the exemplary ventral plane videography methods disclosed herein). The present invention also provides for administration of a beta-adrenergic blocking agent (beta-blocker) to a subject at risk of developing ALS (e.g., a SOD1 G93A mouse) and/or having early stages of ALS, to modulate supranormal gait characteristics and to prevent, treat and/or ameliorate the onset, advancement, severity or effects of a neurodegenerative disease, e.g., ALS, in the subject.

### RELATED APPLICATIONS

This application claims the benefit of U.S. Ser. No. 60/735,389, entitiled “Method for Preventative Treatment of ALS,” filed on Nov. 11, 2005, and U.S. Ser. No. 60/702,377, entitiled “Method for Preventative Treatment of ALS,” filed on Jul. 25, 2005. The entire contents of these applications are hereby incorporated herein by reference.

### BACKGROUND OF THE INVENTION

Amyotrophic lateral sclerosis (ALS), sometimes called Lou Gehrig's disease, is a clinically severe and progressively fatal neurodegenerative disorder characterized by a loss of both upper and lower motor neurons, resulting in progressive muscle wasting and subsequent paralysis (Rowland et al. N. Engl. J. Med. 2001 344:1688-1700). Motor neurons are nerve cells located in the brain, brainstem, and spinal cord that connect the nervous system to voluntary muscles of the body. In ALS, the motor neurons degenerate or die, causing the muscles they enervate to gradually weaken, atrophy, and twitch (fasciculation). Eventually, the ability of the brain to control voluntary movement is lost. When muscles in the diaphragm and chest wall fail, patients lose the ability to breathe without ventilatory support, resulting in death due to respiratory failure. This usually occurs within 3 to 5 years from the onset of symptoms.

The incidence of ALS is approximately 2/100,000/year and may be rising. As many as 20,000 Americans have ALS, and an estimated 5,000 people in the United States are diagnosed with the disease each year. ALS is one of the most common neuromuscular diseases worldwide, and people of all races and ethnic backgrounds are affected. ALS most commonly strikes people between 40 and 60 years of age, but younger and older people can also develop the disease, with men more often affected than women. In 90 to 95 percent of all ALS cases, the disease occurs apparently at random with no clearly associated risk factors. Patients typically do not have a family history of the disease, and their family members are not considered to be at increased risk for developing ALS.

Current medical care for ALS focuses on symptom management. Supportive care ameliorates symptoms and makes ALS more manageable for patients and their families but does not affect the primary disease process. Riluzole, the only FDA-approved ALS therapy, is associated with only a 2-3 month prolongation of survival (Bensimon et al. N. Eng. J. Med. 1994 330:585-591; Miller et al. Amyotroph. Lateral Scler. Other Motor Neuron Disord. 2003 4:191-206). Riluzole is believed to reduce the damage to motor neurons by decreasing the release of glutamate; riluzole does not reverse the damage already done to motor neurons. Because riluzole causes liver damage and has other possible side effects, patients administered the drug must be closely monitored. While certain therapies for the treatment of ALS show promise, the benefits of improved diagnosis of ALS, including improved diagnosis of a subject's predisposition to develop ALS, and of discovery of new therapies that delay disease onset and/or extend patient survival, would be significant.

### SUMMARY OF THE INVENTION

The present invention is based, at least in part, on the discovery that improved methods of examining gait dynamics in a subject can enhance early diagnosis of neurodegenerative disease in the subject. Accordingly, the present invention provides methods of improved early diagnosis of neurodegenerative disease, e.g., ALS, in a subject via measurement of the gait dynamics of the subject (e.g., via use of ventral plane videography to observe a subject on a treadmill as described herein).

The present invention is additionally based, at least in part, on the surprising and unexpected discovery that beta-adrenergic blocking agents (beta-blockers), which are used to treat hypertension, etc., can be used for preventing the onset of neurodegenerative disorders, e.g., amyotrophic lateral sclerosis (ALS), and for treating early stages, including presymptomatic stages, of such neurodegenerative disorders. Accordingly, the present invention provides methods of administration of a beta-adrenergic blocking agent (beta-blocker) to a subject (e.g., a SOD1 G93A mouse) having, at risk of developing, or genetically, metabolically, or environmentally predisposed to develop degenerative symptoms of a neurodegenerative disease, e.g., ALS degenerative symptoms, in order to modulate gait characteristics and also prevent, treat and/or ameliorate the onset, advancement, severity or effects of the neurodegenerative disease, e.g., ALS, in the subject.

In one aspect, the present invention provides a method for preventing early stages, including presymptomatic stages, of amyotrophic lateral sclerosis (ALS) in a subject by administering a beta-adrenergic blocking agent (beta-blocker) to the subject.

In some embodiments, the subject has increased stride lengths in comparison to a standardized average length stride. Whereas a subject diagnosed with ALS using traditional diagnostic criteria can exhibit a reduced average stride length, the present invention identifies that a subject not yet diagnosed with ALS, but genetically, metabolically, or environmentally predisposed to develop ALS degenerative symptoms will likely exhibit presymptomatically increased stride length.

In some embodiments, the beta-blocker is selected from the group consisting of acebutolol, atenolol, betaxolol, bisoprolol, carteolol, celeprolol, labetalol, metoprolol, nadolol, nebivolol, oxprenolol, penbutolol, pindolol, propranolol, sotalol, esmolol, carvedilol, timolol, bopindolol, medroxalol, bucindolol, levobunolol, metipranolol, celiprolol and propafenone. In some embodiments, the beta-blocker is administered parenterally, e.g., intravenously, intradernally, subcutaneously, intraperitoneally, intramuscularly, orally (e.g., by ingestion or inhalation), transdermally (topically) or transmucosally. In certain embodiments, the beta-blocker is orally administered to the subject. In an additional embodiment, the beta-blocker is parenterally administered to the subject.

Another aspect of the invention provides a method of inhibiting adrenergic beta receptor signaling in a subject having early ALS including administering a beta-blocker to the subject.

A related aspect of the invention provides a method of treating or reducing the advancement, severity or effects of ALS in a subject in need thereof by administering a beta-blocker to the subject.

An additional aspect of the invention provides a method of preventing the onset of ALS symptoms, e.g., gait dynamic, neural and/or muscular symptoms, in a subject having ALS including administering an agent which reduces one or more of excitability of a motor neuron, motor performance, and muscle strength.

In one embodiment of the present invention, the agent administered to the subject is a beta-blocker.

Another aspect of the invention provides a method for preventing amyotrophic lateral sclerosis (ALS) in a subject in need thereof by administering a beta-blocker to the subject. In an exemplary embodiment, the beta-blocker is propranolol.

An additional aspect of the invention provides a method of diagnosing early amyotrophic lateral sclerosis (ALS) in a subject including measuring a stride length of the subject and determining whether the stride length of the subject is increased in comparison to a standardized average length stride, wherein an increase in the stride length is indicative of the subject having early ALS.

In one embodiment of the present invention, the stride length of the subject is measured while the subject ambulates on a treadmill.

A further aspect of the present invention provides a method of identifying an agent which treats or reduces the advancement, severity or effects of ALS by administering the agent to an experimental vertebrate predisposed to having ALS or showing ALS symptoms, measuring the stride length of said vertebrate, and determining whether a stride length of the subject is decreased in comparison to a control vertebrate that has not been administered the agent, wherein a decrease in the stride length of the experimental vertebrate indicates the agent treats or reduces the advancement, severity or effects of ALS.

In one embodiment of the present invention, the experimental vertebrate is a rodent. In certain embodiments, the experimental vertebrate is a mouse. In a related embodiment, the mouse is an SOD1 mouse. In certain embodiments, a stride length is measured by placing the experimental vertebrate on a treadmill. In some embodiments, a stride length is measured using ventral plane videography. In an exemplary embodiment, the invention provides a pharmaceutical composition including the identified agent that treats or reduces the advancement, severity or effects of ALS and a pharmaceutically acceptable carrier.

In one embodiment, the invention provides a method of treating a neurodegenerative disease (e.g., ALS) that further comprises administering the subject a compound capable of preventing weight loss.

In another aspect, the invention provides a method of preventing, delaying, or mitigating the symptoms of ALS in a subject in need thereof by administering a beta-blocker and a compound capable of preventing weight loss to the subject.

In certain embodiments of the present invention, the method additionally involves administering an antioxidant to the subject.

A further aspect of the invention provides a method of preventing, delaying, or mitigating the symptoms of ALS in a subject in need thereof by administering propranolol to a subject having or at risk for having ALS, wherein propranolol is formulated in apple juice.

An additional aspect of the invention provides a method of predicting whether a subject is at risk of developing a neurodegenerative condition including determining a foot placement angle variability of the subject and comparing the determined foot placement angle variability to a control foot placement angle variability, wherein an increase in the foot placement angle variability of the subject indicates the subject is at risk for developing a neurodegenerative condition.

In one embodiment of the present invention, the neurodegenerative condition is ALS.

Another aspect of the invention provides a method of identifying an agent which treats or reduces the advancement, severity or effects of a neurodegenerative disease including administering the agent to an experimental vertebrate predisposed to having the neurodegenerative disease or showing signs of the neurodegenerative disease; measuring a foot placement angle variability of said vertebrate; and determining whether the foot placement angle variability of the vertebrate is decreased in comparison to a control vertebrate that has not been administered said agent, wherein a decrease in the foot placement angle variability of the experimental vertebrate indicates the agent treats or reduces the advancement, severity or effects of the neurodegenerative disease.

In one embodiment, the neurodegenerative disease is ALS. In another embodiment, the invention provides a pharmaceutical composition including the identified agent that treats or reduces the advancement, severity or effects of a neurodegenerative disease and a pharmaceutically acceptable carrier.

In another aspect, the invention provides a method of testing a subject to determine whether the subject has or is at risk for developing a neurodegenerative disease including having said subject move on said subject's forelimbs and hindlimbs on a surface; measuring the distance between the subject's forelimbs and hindlimbs; and comparing the measured distance to a control distance, wherein an increased difference between the measured distance compared to the control distance indicates that said subject may have, or is at risk of developing, a neurodegenerative disease.

In a further aspect, the invention provides a method of testing a subject to determine whether the subject has, or is at risk for developing, a neurodegenerative disease including having said subject move on said subject's limbs on a surface; measuring one or more angles made by said subject's limbs relative to the centerline of said subject's body; and comparing the measured angles to control angles, wherein an increased difference between one or more measured angles compared to control angles indicates that said subject may have, or is at risk for developing, a neurodegenerative disease.

In another aspect, the invention provides a predictive method of determining whether a subject has or is at risk for developing a neurodegenerative disease including measuring the limb placement variability and a stance width of said subject, wherein an increase in both measurements relative to a suitable control indicates said subject has, or is at risk for developing, a neurodegenerative disease.

In one embodiment, the invention provides a method of treating a subject identified as having a neurodegenerative disease using any one of the methods of the invention, including administering propranolol to said subject. In another embodiment, the invention provides a method of preventing a neurodegenerative disease in a subject identified using any one of the methods of the invention, including administering propranolol to said subject.

Another aspect of the present invention provides a method for preventing or treating a neurodegenerative disease in a subject including administering an antioxidant to the subject.

In an exemplary embodiment, the antioxidant is isolated 4-HO-propranolol (4HOP).

An additional aspect of the present invention provides kits for treating, preventing or diagnosing ALS in a subject that includes a beta-blocker and instructions for use. In certain embodiments of the present invention, the kit additionally includes an antioxidant. In one embodiment, the propranolol is included in a sport beverage, such as Gatorade, or included in a diet for military subjects, among whom there is an unusually high incidence of ALS. Another aspect of the present invention provides a method for preventing or delaying the onset of ALS in a subject with supranormal gait via administration of a beta-blocker (e.g., propranolol) to the subject. A further aspect of the present invention provides a method for preventing or delaying the onset of ALS in a subject having an increased stride length in comparison to a standardized average length stride via administration of a beta-blocker (e.g., propranolol) to the subject.

Other features and advantages of the invention will be apparent from the following detailed description and claims.

### BRIEF DESCRIPTION OF THE DRAWINGS

FIG. 1A shows two images depicting the ventral view of a saline-treated C57BL/6J mouse on a transparent treadmill belt walking at a speed of 34 cm/s. The image on the left depicts full stance for the right hind limb, and the image on the right depicts sequential full stance for the left hind limb. Cartesian coordinates are used to determine stance width and paw placement angles for the forelimbs and hind limbs. FIG. 1B depicts representative gait signals of the left forelimb and right hind limb of a saline-treated C57BL/6J mouse walking at a speed of 34 cm/s. Duration of stride, stance, and swing are indicated for the left fore paw. Duration of braking and propulsion are indicated for the right hind paw. FIG. 2 demonstrates that drugs can induce alterations in gait, and such alterations in gait are associated with a movement disorder. Gait signals of the right hind limb of a MPTP-treated mouse superimposed over gait signals of the right hind limb of a saline-treated mouse are shown. Stride frequency was higher, while stance duration and swing duration were shorter, in MPTP-treated mice compared to saline-treated mice. MPTP is a neurotoxin that induces Parkinsonian symptoms.

FIG. 3 demonstrates that drugs (e.g., MPTP and 3NP) can induce alterations in gait, and such alterations in gait are associated with a movement disorder. Further demonstrated is that gait alterations can be distinct between different types of movement disorders (e.g., Huntington's disease symptoms in mice compared to Parkinson's symptoms in mice). Stride time (gait cycle duration) dynamics of MPTP-treated, 3NP-treated, and saline-treated mice are shown. Right forelimb measurements are shown in left panels, while left hind limb measurements are shown in right panels. In saline-treated animals, forelimb stride variability was higher than hind limb stride variability. MPTP-treated and 3NP-treated mice exhibited significantly higher stride variability. The coefficient of variation (CV), a measure of stride-to-stride variability, was highest in the forelimbs of 3NP-treated mice. 3NP is a neurotoxin that induces symptoms in mice comparable to Huntington's disease symptoms in humans.

FIG. 4A shows the ventral view of a 3NP-treated mouse attempting to walk on the treadmill belt moving at a speed of 34 cm/s but failing to engage the hind limbs in coordinated stepping. This animal braced its hind paws onto the base of the sidewalls of the running compartment avoiding the moving treadmill belt. Only the forelimbs executed coordinated stepping sequences. FIG. 4B depicts gait signals of the left and right forelimbs of a 3NP-treated mouse demonstrating coordinated stepping, despite hind limb failure of stepping. The signals of left and right hind limbs were not coordinated and reflect artefacts associated with the belt contacting the braced paws.

FIG. 5 shows a ventral view of a mouse, depicting measurement of stance width and paw placement angle values.

FIGS. 6A and 6B graphically depict forelimb mean paw angles and forelimb paw angle variability for ALS mice (SOD1 G93A mice) walking on a treadmill at 34 cm/s (squares) and 23 cm/s (circles).

FIGS. 7A and 7B graphically depict hind paw mean paw angles and hind paw angle variability for ALS mice walking on a treadmill at 34 cm/s (squares) and 23 cm/s (circles).

FIGS. 8A and 8B graphically depict the impact of propranolol treatment of ALS mice (SOD1 G93A mice) on body weight and survival (propranolol-treated mice are represented by squares, while control tap water-treated mice are represented by triangles or diamonds).

### DETAILED DESCRIPTION OF THE INVENTION

The present invention is based, at least in part, on the surprising and unexpected discovery that beta-adrenergic blocking agents (beta-blockers), which are used to treat hypertension, etc., can be used for treating early stages, including presymptomatic stages, of neurodegenerative disorders, e.g., amyotrophic lateral sclerosis (ALS). Specifically, it was observed that administration of a beta-adrenergic blocking agent (beta-blocker) to a subject having or at risk of developing ALS (e.g., a SOD1 G93A mouse) can both reduce supranormal gait characteristics and also prevent, treat, delay, mitigate and/or ameliorate the onset, advancement, severity and/or symptoms of a neurodegenerative disease, e.g., ALS.

The present invention is the first to describe the use of propranolol to mitigate or prevent gait “supranormalcy” seen presymptomatically (e.g, absent neurodegenerative symptoms) in subjects that develop ALS, and is also the first to identify the use of propranolol to mitigate or prevent the gait disturbances that are present with and after ALS is diagnosed in a subject (during early stage ALS disease). Currently, propranolol is administered to ALS patients to treat sialorrhea via reduction of thick mucus production associated with ALS. The present invention is based, at least in part, upon the surprising discovery that administration of propranolol to an ALS and/or ALS-predisposed subject without mucous tissue-related symptoms (mucosal involvement) can effectively prevent or delay the onset of neurodegenerative symptoms of ALS and/or can effectively mitigate and/or treat such symptoms of ALS.

Accordingly, the invention provides methods for preventing, delaying onset or progression and/or otherwise treating a neurodegenerative disease or disorder (e.g., ALS) in a subject via administration of a beta-blocker to a subject having, or at risk of developing, a neurodegenerative disease or disorder (e.g., ALS).

The present invention is also based, at least in part, on the surprising discovery of supranormal gait dynamics in subjects having, or at risk of developing, ALS (e.g., SOD1 G93A mice), as observed via measurement (e.g., via ventral plane videography) of a subject's gait on a treadmill. Such supranormal gait was observed in ALS subjects during a time interval prior to complete neurodegenerative progression of ALS. Thus, it was determined that improved methods of examining gait dynamics in a subject can enhance early diagnosis of neurodegenerative disease in the subject. Accordingly, the present invention, at least in part, provides methods of improved early diagnosis of neurodegenerative disease, e.g., ALS, via measurement of the gait dynamics of a subject (e.g., via the exemplary ventral plane videography methods disclosed herein). Specifically, increased stride length in a subject walking on a treadmill in comparison to the stride length of another subject walking on a treadmill at equal or comparable walking speed can be an indicator of presymptomatic propensity for a subject to develop ALS degenerative characteristics. Assessment of overground gait dynamics in a subject is likely a less robust method of providing this diagnosis, as compared to treadmill locomotion and gait analysis of a subject on a treadmill, which can provide early indication of ALS via determination of increased stride length on a treadmill. (However, it is envisioned that under conditions where walking speeds of overground walkers were equal or comparable to treadmill walkers, the diagnosis could also be made.) The advantage of the treadmill is better control and/or pre-selection of the walking speed to eliminate differences in walking speed as a confounder in the comparison.

One exemplary beta-blocker is propranolol. Another exemplary beta-blocker is an art-recognized metabolite of propranolol, 4-HO-propranolol (4HOP). In view of the documented antioxidant properties of 4HOP, certain aspects of the present invention provide methods for preventing or treating a neurodegenerative disease in a subject via administration of an antioxidant. Accordingly, the invention provides for administration of beta-blocker and/or antioxidant agents alone or in combination to a subject.

Exemplary routes of administration for the beta-blocker include parenteral, e.g., intravenous, intradermal, subcutaneous, intraperitoneal, intramuscular, oral (e.g., by ingestion or inhalation), transdermal (topical) or transmucosal. The present invention also provides for oral administration of a beta-blocker, e.g., propranolol, formulated in apple juice. Accordingly, certain aspects of the invention provide methods of preventing, delaying, or mitigating the symptoms of ALS in a subject having or at risk of developing ALS via administration of propranolol forumulated in apple juice to a subject. (Among other effects, formulation of propranolol in apple juice can make the ingestion of such a formulation more pleasurable.) One of ordinary skill in the art will recognize that otherjuices, liquids, and/or beverages can be used to dissolve the agents of the present invention. Such juices, liquids and/or beverages can provide for enhanced delivery of an agent to a subject, and can also impart a preventive and/or therapeutic effect that enhances the effect of the forumulated agent. The present invention additionally provides for administration of agents forumulated in fruit- and/or vegetable-derived juices containing antioxidants (or in other liquids containing antioxidants) to a subject having, or at risk of developing, ALS.

Supranormal gait in early ALS and/or ALS-predisposed subjects is likely reflective of an enhanced excitability of motor neurons, motor performance, and/or muscle strength in these subjects. Accordingly, the present invention also provides a method of preventing the onset of ALS symptoms in a subject having early ALS via administration of an agent which reduces excitability of motor neurons, motor performance, and/or muscle strength to the subject. Exemplary excitability indices studied can include, e.g., stimulus-response curve (SR); strength-duration time constant (tau(SD)); current/threshold relationship; threshold electrotonus to a lOOms polarizing current; and recovery curves to a supramaximal stimulus (Vucic, S. et al. Clin Neurophysiol. 2006 July; 117(7):1458-1466. Epub 2006 Jun. 8). Kanai et al. recently observed axonal excitability as altered in ALS patients (Brain 2006 April;129(Pt 4):953-62. Epub 2006 Feb. 8). Exemplary indices of motor performance include tests of balance and gait, and standing up from a sitting position. Exemplary indices of muscle strength include hand strength, arm strength, leg strength, tongue strength, or any muscle group. Other measurements include timed functional activities, and isometric strength using an electronic strain gauge. In rodents, exemplary muscle strength indices include grip strength, as measured by the ability of a paw to grasp a wire or rod as the body of the animal is tugged to cause the animal to release its grip on said wire or rod; briefer times indicate weaker strength; longer times to release indicate stronger strength.

Without wishing to be bound by theory, it is contemplated that other suitable means for diagnosing presymptomatic ALS may also be used in the methods of the invention such as, for example, MRI, EMG, etc.

The present invention additionally provides a method of inhibiting adrenergic beta receptor signaling in a subject having early ALS via administration of a beta-blocker to the subject.

The present invention also provides a method for identifying an agent which treats or reduces the advancement, severity or effects of ALS, via administration of the agent to an experimental vertebrate predisposed to have ALS or show symptoms of early ALS, measurement of an index or indices of gait dynamics associated with predisposition to and/or progression of ALS (e.g., stride length, paw or foot placement angle variability), and determination of whether the gait dynamics index or indices are decreased in comparison to a control vertebrate that has not been administered the agent. The invention additionally provides a method for identifying an agent which treats or reduces the advancement, severity or effects of a neurodegenerative disease, via administration of the agent to an experimental vertebrate predisposed to have the neurodegenerative disease or showing signs of the neurodegenerative disease, measurement of foot placement angle variability in the vertebrate, and determination of whether the foot placement angle variability of the vertebrate is decreased in comparison to a control vertebrate that has not been administered the agent. Such screening methods may be performed using any agent. Representative assemblages of test agents/compounds are described below.

Other aspects of the invention provide methods for diagnosing neurodegenerative disease or a predisposition to develop neurodegenerative disease in a subject. One such aspect provides a method of diagnosing early amyotrophic lateral sclerosis (ALS) in a subject via measurement of the stride length of the subject and determination of whether the stride length of the subject is increased in comparison to a standardized average length stride. Another such aspect provides a method of predicting whether a subject is at risk of developing a neurodegenerative condition via determination of the foot placement angle variability of the subject and comparison of the determined foot placement angle variability to a control foot placement angle variability, with an increase in the foot placement angle variability of the subject indicating that the subject is at risk for developing a neurodegenerative condition. An additional aspect provides a method of testing a subject to determine whether the subject has or is at risk of developing a neurodegenerative disease by having the subject crawl on her hands and knees on a surface, measuring the distance between the subject's hands and knees; and comparing the measured distance to an appropriate control distance, with an increased difference in the measured distance compared to the control distance indicating that the subject may have, or is at risk of developing, a neurodegenerative disease. A further aspect provides a method of testing a subject to determine whether the subject has or is at risk of developing a neurodegenerative disease by having the subject crawl on his hands and knees on a surface, measuring the angles made by the subject's hands and knees relative to the centerline of the subject's body; and comparing the measured angles to appropriate control angles, with an increased difference in the measured angle as compared to the control angle indicating that the subject may have, or be at risk of developing, a neurodegenerative disease.

Measurement of certain gait indices may also be combined. Accordingly, an additional aspect of the invention provides a predictive method of determining whether a subject has, or is at risk of developing, a neurodegenerative disease by measurement of the limb placement variability and the stance width of the subject, with an increase in both measurements indicating that the subject has, or is at risk of developing, a neurodegenerative disease.

In certain embodiments, the present invention provides a method for treating a patient diagnosed with or at risk for developing ALS, involving administering a compound (e.g., propranolol), in an amount sufficient to treat the patient. The amount of compound (e.g., beta-blocker, e.g., propranolol) administered can be determined by one skilled in the art, but should be an amount sufficient to treat the symptoms of ALS and/or prevent, reduce and/or inhibit the progression of ALS in the subject, relative to a subject that is not treated with the compound.

An effective amount of active compound(s) used to practice the present invention for therapeutic treatment of ALS varies depending upon the manner of administration, the age, body weight, and general health of the patient. Ultimately, the attending physician or veterinarian will decide the appropriate amount and dosage regimen. Such amount is referred to as an effective, sufficient, or therapeutic amount.

In certain embodiments, the administration of compound (e.g., beta-blocker compound) results in a delay in ALS disease progression of at least one day, relative to control subjects, and may be more than one week, one month, three months, six months, one year, five years, etc.

The pharmaceutical compositions of the instant invention can be included in a kit, in a container, pack, beverage and beverage container, sports drink, or dispenser together with instructions for administration.

So that the invention may be more readily understood, certain terms are first defined.

I. Definitions

As used herein, the term “gait” refers to a sequence of paw/foot or limb movements by which a subject (e.g., a human, mouse or other animal) moves, or attempts to move, in a directional manner. In exemplary usage, the direction of movement is forward. Also in exemplary usage, the term “gait” refers to a rhythmic and/or cyclical ambulatory process performed by at least one limb of a subject; however, the rhythm and/or cyclicality of a subject's ambulatory process can be highly disrupted, with the process still properly characterized as “gait”.

As used herein, the term “stride length” refers to the distance traveled during one cycle of gait (e.g., the distance traveled between the point at which a foot, paw, knee, hand, etc. of a moving (e.g., ambulating) subject departs contact with a primary supporting surface (e.g., the ground or other walking surface) and the point at which the same foot, paw, knee, hand, etc. of the subject next contacts the supporting surface. As used herein, the term “standardized average stride length” refers to the average measured value for stride length observed for a population that has not been selected for, or is not anticipated to have been selected for, a disease, disorder or any other attribute that alters, or would be anticipated to alter, the measured average stride length of the population.

As used herein, the term “early ALS” refers to both the period of time preceding the development of ALS symptoms in a subject and the initial period of ALS disease progression in a subject during which the subject with ALS retains sufficient control over motor neurons and sufficient voluntary limb mobility to allow for gait to be measured in the subject while ambulating on a surface. Accordingly, a subject with “early ALS” may display no symptoms of ALS, or may display any range of symptoms of ALS that do not prevent the subject from performing a voluntary ambulatory motion. Early ALS, as used herein, is also characterized by absence of severe mucous tissue involvement that characterizes later stages of the disease, e.g., absence of a condition of thick mucus production (e.g., siallorhea) in such subjects. ALS is a progressive disease that causes increasing muscle weakness, inability to control movement, and problems with speaking, swallowing, and breathing in a subject. Early signs of degenerative ALS include slight weakness in one leg, one hand, the face, or the tongue of a subject. Other signs of early ALS can include increasing clumsiness and difficulty performing tasks that require precise movements of the fingers and hands. Frequent muscle twitching can occur during early ALS. As ALS progresses, the weakness slowly spreads to the arms and legs over a period of time (e.g., months or years). As motor nerves continue to waste away and decrease in number, the muscle cells that would normally be stimulated by those nerves also start to waste away, and the muscles weaken. A subject who has lost voluntary limb mobility to the extent that gait on a surface may no longer be measured has progressed beyond the stage of “early ALS” for purposes of the present invention.

As used herein, the term “beta-blocker” refers to an agent that binds to a beta-adrenergic receptor and inhibits the effects of beta-adrenergic stimulation. Beta-blockers typically increase AV nodal conduction. In addition, beta-blockers have been reported to decrease heart rate by blocking the effect of norepinephrine on the post synaptic nerve terminal that controls heart rate. Beta blockers have also been reported to decrease intracellular Ca⁺⁺ overload, which inhibits after-depolarization mediated automaticity. Exemplary beta blockers include, but are not limited to, for example, acebutolol (Sectral), atenolol (Atenix, Antipressan, Tenormin), betaxolol (Kerlone), bisoprolol (Cardicor, Emcor, Monocor, Zebeta), carteolol (Cartrol), celeprolol (Celectol), labetalol (Normodyne, Trandate), metoprolol (Mepranix, Betaloc, Lopressor), nadolol (Corgard), nebivolol (Nebilet), oxprenolol (Trasicor), penbutolol, pindolol (Visken), sotalol (Beta-cardone, Sotacor), esmolol (Brevibloc), carvedilol (Eucardic), timolol (Betim), bopindolol (Sandonorm), medroxalol, bucindolol, levobunolol (Betagan), metipranolol (OptiPranolol), celiprolol (Selectol), propafenone (Rythmol), propranolol (Propanix, Angilol, Inderal) and 4-HO-propranolol (4HOP, a propranolol metabolite); alternative pharmaceutically acceptable salts, esters, hydrates, complexes, etc. of these compounds; and/or compounds which are competitive antagonists of a beta-adrenergic receptor to a degree which is at least about 25% (e.g., at least about 50%, at least about 75%, at least about 100%) that of propranolol. Combinations, derivatives and metabolites of various beta blockers can also be employed, and the term “beta blocker” is meant to include such combinations of beta blockers.

A beta blocker used in the method of the present invention can be administered alone or in combination with suitable pharmaceutical carriers or diluents. Diluent or carrier ingredients used in the beta blocker formulation should be selected so that they do not diminish the desired effects of the beta blocker. A beta blocker formulation may be made up in any suitable form appropriate for the administration to a subject. Examples of suitable dosage forms include solutions, and the like. In certain embodiments, the beta-blocker is formulated in a liquid, e.g., water, apple juice, grape juice, berry juice, etc., that may be orally administered to a subject. Alternatively, a beta blocker can be provided in the form of a sterile solid composition which can be forumulated in a sterile injectable medium immediately before use. Suitable beta blocker formulations include those which contain other excipients known in the art, such as those further discussed below.

The beta blocker, depending on the vehicle and concentration used, can be forumulated in the vehicle in any suitable concentration. In preparing solutions, the beta blocker can be forumulated in saline and filter sterilized before filling into a suitable vial or ampule and sealing. Advantageously, adjuvants, such as preservatives and buffering agents, can be forumulated in the vehicle. To enhance the stability, the composition can be freeze-dried. The dry lyophilized powder can then sealed in the vial, and an accompanying vial of water for injection can be supplied to reconstitute the liquid prior to use.

In addition to the above-described excipients etc., the beta blocker formulation can also (i.e., in addition to the beta blocker) contain other active pharmaceutical agents, such as those discussed below.

Exemplary beta blocker agents of the invention can be administered at a variety of concentrations, and exemplary administered concentration ranges of beta blocker in the beta blocker formulation can depend upon the route of administration and/or partition coefficients of such formulations, as is recognized by one of ordinary skill in the art in the field of pharmacology. Accordingly, exemplary beta blockers of the invention can be administered in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg. Parenteral administration of exemplary compounds can occur over any suitable concentration range, including from about 0.1 mg/ml to about 10 mg/ml, such as from about 0.5 mg/ml to about 2 mg/ml and/or about I mg/ml. In exemplary embodiments, 0.5 g/L of propranolol is added to the drinking water of a subject. In certain embodiments, mouse subjects are administered about 5 mg propranolol per about 20 gram mouse per day. In other embodiments, human subjects are administered about 20 grams/day. Accordingly, exemplary beta blockers of the invention can also be administered in the range of from about 1 g to about 500 g, about 5 g to about 450 g, about 6 g to about 400 g, about 7 g to about 350 g, about 8 g to about 300 g, about 9 g to about 250 mg, about 10 g to about 200 g, about 11 g to about 150 g, about 12 g to about 100 g, about 13 g to about 50 g, about 14 g to about 45 g, about 15 g to about 40 g, about 16 g to about 35 g, about 17 g to about 30 g, about 18 g, about 19 g, about 20 g, about 21 g, about 22 g, about 23 g, about 24 g, about 25 g, about 26 g, about 27 g, about 28 g, or about 29 g.

Suitable dosages can be ascertained by standard methods, such as by establishing dose-response curves in laboratory animal models or in clinical trials. Illustratively, suitable dosages of an injectable beta blocker (administered in a single bolus or over time) include from about 1 μg/kg (of the subject's body weight) to about 150 μg/kg, such as from about 3 μg/kg to about 75 μg/kg, from about 5 μg/kg to about 50 μg/kg, from about 10 μg/kg to about 25 μg/kg, and/or about 15 μg/kg.

The term “antioxidant” or “anti-oxidant” includes chemical compounds that can absorb an oxygen radical, e.g., ascorbic acid and phenolic compounds. The term “antioxidant activity” refers to a measurable level of oxygen radical scavenging activity, e.g. the oxygen radical absorbance capacity (ORAC) of an extract, fraction, or compound. The term “antioxidant responsive condition” includes any disease or condition that is associated with the presence of undesired oxidation, oxygen radicals, or other free radicals.

As used herein, the term “treating” includes the administration of a pharmaceutical composition for the treatment or prevention of ALS. To “treat disease” or use for “therapeutic treatment” refers to administering treatment to a patient already suffering from ALS to improve the patient's condition (i.e., to reduce or prevent motor neuron degeneration, preserve motor neuron function, and maintain a patient's normal lifestyle). The term “patient” means any animal (e.g., a human).

As used herein, the term “compound” includes any reagent which is tested using the methods of the invention to determine whether it modulates ALS progression. More than one compound, e.g., a plurality of compounds, can be tested at the same time for their ability to modulate ALS progression in a screening assay.

As used herein, the term “oxidant stress” encompasses the perturbation of the ability of a cell to ameliorate the toxic effects of oxidants. Oxidants may include hydrogen peroxide or oxygen radicals that are capable of reacting with bases in the cell including DNA. A cell under oxidant stress may undergo biochemical, metabolic, physiological and/or chemical modifications to counter the introduction of such oxidants. Such modifications may include lipid peroxidation, NF-kB activation, heme oxygenase type I induction and DNA mutagenesis. Also, antioxidants such as glutathione are capable of lowering the effects of oxidants. “Cellular stress” may also be induced by serum starvation or by the withdrawal or deprivation of other trophic factors which may perturb normal cellular function. Such perturbations may be by the same or by different mechanisms as that induced by oxidant stress.

As used herein, the term “neuronal degeneration” or “neurodegeneration” encompasses a decline in normal functioning of a neuronal cell. Such a decline may include a decline in memory, learning, perception, neuronal electrophysiology (i.e., action potentials, polarizations and synapses), synapse formation, both chemical and electrical, channel functions, neurotransmitter release and detection and neuromotor functions. In the present invention, the subject may be a mammal or a human subject.

As used herein, the term “compound capable of preventing weight loss” or “weight loss inhibitor” refers to any agent capable of reducing and/or preventing the wasting phenotype that commonly accompanies progression of many neurological diseases, e.g., ALS. Such agents can include dietary supplements, e.g., high fat and/or high calorie agents. Such agents can also include, e.g., conjugated linoleic acids and other agents associated with reduction and/or prevention of weight loss in a subject (e.g., a human, mouse, rat or other animal). Such agents can also include those possessing an above-average level of olfactory and/or flavor interest to a subject.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined for an organism, e.g., a control or normal organism, exhibiting, for example, normal traits. In yet another embodiment, a “suitable control” or “appropriate control” is a predefined value, level, feature, characteristic, property, etc.

The experimental vertebrate that may be used in screening methods of the invention can be any vertebrate which includes at least one forelimb, and preferably at least three total limbs. Exemplary vertebrates useful in the methods described herein include, but are not limited to, rats, mice, hamsters, guinea pigs, cats, and dogs. In one embodiment, an experimental vertebrate useful in the methods of the invention is a rodent. Exemplary rodents that may be used in the screening methods of the invention include rats, mice, gerbils, hamsters, cavies, guinea pigs, and chinchillas.

Various aspects of the invention are described in further detail in the following subsections.

II. Gait Measurement and Neurological Disease

Mouse Models of Neurological Disease

Gait abnormalities are characteristic and symptomatic of Parkinson's disease (PD), Huntington's disease (HD), and amyotrophic lateral sclerosis (ALS). Gait reflects several variables, including balance, proprioception, and coordination. There are several mouse models of PD (Sedelis et al. Behav Brain Res 2001, 125: 109-125; Fleming et al. J Neurosci 2004, 24: 9434-9440) and HD (Santamaria et al. Neurochem Res 2001, 26: 419-424; Lin et al. Hum Mol Genet 2001, 10:137-144; Carter et al. J Neurosci 1999, 19: 3248-3257; Naver et al. Neuroscience 2003, 122: 1049-1057), and one widely studied model of ALS (Gurney et al. Science 1994, 264: 1772-1775; Fischer et al. Exp Neurol 2004, 185: 232-240; Puttaparthi et al. J Neurosci 2002, 22: 8790-8796; Bameoud et al. Neuroreport 1997, 8:2861-2865). Mouse models that replicate PD, HD, and ALS symptoms can improve understanding of pathogenesis, prognosis and treatment of these diseases.

Gait abnormalities in PD include shortened stride length (Salarian et al. IEEE Trans Biomed Eng 2004, 51: 156-159; Weller et al. Br J Clin Pharmacol 1993, 35: 379-385), a dyscontrol of stride frequency (Bartolic et al. Eur J Neurol 2005, 12: 156-159), and postural instability (Nieuwboer et al. Mov Disord 2001, 16: 1066-1075). Gait abnormalities in HD include reduced walking speed (Thaut et al. Mov Disord 1999, 14: 808-819), widened stance width (Koller et al. Neurology 1985, 35: 1450-1454), reduced stride length (ibid; Bilney et al. Mov Disord 2005, 20: 51-57), and sway (Tian et al. Neurology 1992, 42: 1232-1238). Gait variability has also been shown to be significantly higher in patients with PD (Hausdorff et al. Mov Disord 1998, 13: 428-437; Blin et al. J Neurol Sci 1990, 98: 91-97; Schaafsma et al. J Neurol Sci 2003, 212: 47-53) and HD (Bilney et al. Mov Disord 2005, 20: 51-57, Hausdorff et al. Mov Disord 1998, 13: 428-437) compared to control subjects. Early detection of gait disturbances may result in earlier treatment. Therapies for PD and HD patients are often developed to ameliorate gait abnormalities (Djaldetti et al. J Neurol 2002, 249 Suppl 2:1130-35, Bonelli et al. Int Clin Psychopharmacol 2004, 19:51-62). Mouse models of PD and HD are used to understand the pathologies of the diseases and to accelerate the testing of new therapies to correct motor defects. Although spatial gait indices have been reported (Fernagut et al. J Neurosci Methods 2002, 113: 123-130; Carter et al. J Neurosci 1999, 19: 3248-3257), gait dynamics in mouse models of PD and HD have not yet been described.

MPTP-Induced Mouse Model of PD

One exemplary mouse model of PD is obtained by repeatedly administering the neurotoxin 1-methyl-4-phenyl-1,2,3,6-tetrahydropyridine (MPTP) (Kopin I J. Environ Health Perspect 1987, 75: 45-51; Sedelis et al. Behav Genet 2000, 30: 171-182; Jakowec et al. Comp Med 2004, 54: 497-513). MPTP causes damage of the nigrostriatal dopaminergic system (Gupta et al. Brain Res Bull 1984, 13: 737-742), resulting in PD symptoms, including reduced stride length (Femagut et al. J Neurosci Methods 2002, 113: 123-130) and posture disturbances in mice (Sedelis et al. Behav Brain Res 2001, 125: 109-125).

3NP-Induced Mouse Model of HD

One exemplary mouse model of HD is obtained by repeatedly administering the mitochondrial toxin 3-nitropropionic acid (3NP) (Schulz et al. Mol Cell Biochem 1997, 174: 193-197; Santamaria et al. Neurochem Res 2001, 26: 419-424). 3NP causes striatal neurodegeneration resulting in mild dystonia and bradykinesia comparable to HD in people (Guyot et al. Neuroscience 1997, 7:45-56, Brouillet et al. Proc Natl Acad Sci U S A 1995, 92: 7105-7109).

Motor defects in MPTP-treated mice or 3NP-treated mice are often quantified using the rotarod test that measures the time a subject can balance on a rotating rod (Diguet et al. Eur J Neurosci 2004, 19: 3266-3276, Dunham et al. J Am Pharm Ass 1957, 46: 208-209). MPTP has been shown to reduce performance on the rotarod (Rozas et al. J Neurosci Methods 1998, 83: 165-175) or to have no effect on rotarod performance (Sedelis et al. Behav Genet 2000, 30: 171-182; Willis et al. Brain Res 1987, 402: 269-274). 3NP has been shown to reduce rotarod performance (Fernagut et al. Neuroscience 2002, 114: 1005-1017), or to have no effect on rotarod performance (Fernagut et al. Eur J Neurosci 2002, 15: 2053-2056). The swim test (Weihmuller et al. Neurosci Lett 1988, 85: 137-42), balance beam test (Ryu et al. Neurobiol Dis 2004, 16: 68-77), and the pole test (Ogawa et al. Res Commun Chem Pathol Pharnacol 1988, 50: 435-441) have also been used to investigate the effects of MPTP and 3NP on motor function in mice. Results regarding motor dysfunction in the MPTP model of PD and the 3NP model of HD may vary due to the heterogeneity in protocols followed. Disparities in the degree of motor dysfunction have suggested that large doses of MPTP or 3NP may be required to detect motor defects after nigrostriatal damage (Jakowec et al. Comp Med 2004, 54: 497-513; Femagut et al. Neuroscience 2002, 114: 1005-1017; Fornai et al. Proc Natl Acad Sci U S A 2005, 2:3413-3418).

Several studies in mouse models of PD and HD have described “gait” by estimating stride length (Fernagut et al. J Neurosci Methods 2002, 113: 123-130), and stance width (Carter et al. J Neurosci 1999, 19: 3248-3257) determined by painting the animals' paws. Fernagut et al. reported that stride length is a reliable index of motor disorders due to basal ganglia dysfunction in mice (Carter et al. J Neurosci 1999, 19: 3248-3257). Gait dynamics in humans, mice and other animals, however, extend beyond the measure of stride length. For example, gait dynamics in both humans and mice include spatial indices such as stance width and foot/paw placement angle. Gait dynamics in humans and mice also include temporal indices, such as stride frequency, stride duration, swing duration, and stance duration. Step-to-step gait variability in humans has also provided important information about possible mechanisms involved in neurodegenerative diseases, including PD and HD (Bilney et al. Mov Disord 2005, 20: 51-57; Hausdorff et al. Mov Disord 1998, 13: 428-437; Blin et al. J Neurol Sci 1990, 98: 91-97; Schaafsma et al. J Neurol Sci 2003, 212: 47-53). In patients with PD, higher step-to-step variability has been reported (Hausdorff et al. Mov Disord 1998, 13: 428-437; Blin et al. J Neurol Sci 1990, 98: 91-97; Schaafsma et al. J Neurol Sci 2003, 212: 47-53; Vieregge et al. J Neural Transm 1997, 104: 237-248). The stride length variability increased with the progression of PD suggesting that this index is useful in assessing the course of PD (Blin et al. J Neurol Sci 1990, 98: 91-97). Hausdorff et al. demonstrated significantly higher variability in several gait indices, including stride duration and swing duration, in patients with PD and HD (Mov Disord 1998, 13: 428-437), and in subjects with amyotrophic lateral sclerosis (ALS) (Hausdorff et al. J Appl Physiol 2000, 88: 2045-2053). It has been proposed that a matrix of gait dynamic markers could be useful in characterizing different diseases of motor control (ibid).

SOD1 Mouse Model of ALS

While the cause of ALS is not known, about 15-20 percent of all cases are familial and result from missense mutations in the enzyme copper/zinc superoxide dismutase (SOD1; Rosen et al. Nature 1993 362:59-62). The similarity in the course and pathological features of familial and sporadic ALS has prompted the view that all forms of the disease may be better understood and ultimately treated by elucidating disease pathogenesis and developing effective therapeutics using transgenic mouse and rat models of ALS expressing mutant forms of SOD1 (Brown et al. Semin. Neurol. 2001 21:131-139; Andersen et al. Amyotroph. Lateral Scier. Other Motor Neuron Disord. 2003 4: 62-73). SOD1 is a powerful antioxidant that protects the body from damage caused by free radicals produced by cells during normal metabolism. It is not clear how this enzyme is involved in ALS, although transgenic mice expressing several of the mutant SOD I genes found in humans with ALS develop motor neuron symptoms and histopathology resembling features of the human disease (Gurney et al. Science 1994 264:1772-1775; Ripps et al. Proc. Natl. Acad. Sci. USA 1995 92:689-693; Bruijn et al. Neuron 1997 18:327-338). A small set of beneficial therapeutic trials in transgenic ALS mice have generated trials of potential treatments in humans with both sporadic and familial ALS (Drachman et al. Ann. Neurol. 2000 52:771-778; Kieran et al. Nat. Med. 2004 10:402-405; Klivenyi et al. Nat. Med. 1999 5:347-350; Zhu et al. Nature 2002 417:74-78).

Improved analyses of gait and stride variability (e.g., quantitative measurement of temporal and spatial indices of gait dynamics) in mouse models of PD, HD and ALS would prove beneficial to the field.

Gait Variability Indices

Gait variability indices are increasingly being recognized as important markers of neurological diseases (Nieuwboer et al. Mov Disord 2001, 16: 1066-1075; Hausdorff et al. Mov Disord 1998, 13: 428-437; Blin et al. J Neurol Sci 1990, 98: 91-97; Schaafsma et al. J Neurol Sci 2003, 212: 47-53; Hausdorff et al. J Appl Physiol 2000, 88: 2045-2053). Several studies in mouse models of PD and HD have described “gait” by estimating stride length (Fernagut et al. J Neurosci Methods 2002, 113: 123-130), and stance width (Carter et al. J Neurosci 1999, 19: 3248-3257) determined by painting the animals' paws. Fernagut et al. reported that stride length is a reliable index of motor disorders due to basal ganglia dysfunction in mice (Carter et al. J Neurosci 1999, 19: 3248-3257). Gait dynamics in humans, mice and other animals, however, extend beyond the measure of stride length. For example, gait dynamics in both humans and mice include spatial indices such as stance width and foot/paw placement angle. Gait dynamics in humans and mice also include temporal indices, such as stride frequency, stride duration, swing duration, and stance duration.

Step-to-step gait variability in humans has also provided important information about possible mechanisms involved in neurodegenerative diseases, including PD and HD (Bilney et al. Mov Disord 2005, 20: 51-57; Hausdorff et al. Mov Disord 1998, 13: 428-437; Blin et al. J Neurol Sci 1990, 98: 91-97; Schaafsma et al. J Neurol Sci 2003, 212: 47-53). In patients with PD, higher step-to-step variability has been reported (Hausdorff et al. Mov Disord 1998, 13: 428-437; Blin et al. J Neurol Sci 1990, 98: 91-97; Schaafsma et al. J Neurol Sci 2003, 212: 47-53; Vieregge et al. J Neural Transm 1997, 104: 237-248). The stride length variability increased with the progression of PD suggesting that this index is useful in assessing the course of PD (Blin et al. J Neurol Sci 1990, 98: 91-97). Hausdorff et al. demonstrated significantly higher variability in several gait indices, including stride duration and swing duration, in patients with PD and HD (Mov Disord 1998, 13: 428-437), and in subjects with amyotrophic lateral sclerosis (ALS) (Hausdorff et al. J Appl Physiol 2000, 88: 2045-2053).

The CVs of stride length and stance width in healthy humans are ˜3-6% and ˜14-17%, respectively (Brach et al. Journal of NeuroEngineering and Rehabilitation 2005, 2: 21; Menz et al. Gait Posture 2004, 20: 20-25). The CV of stride time in humans with intact neural control is <3%, and is significantly higher in patients with PD, HD, and ALS (Hausdorff et al. J Appl Physiol 2000, 88: 2045-2053). Stride time variability was highest in patients with HD (ibid). The CV for stride length in saline-treated C57BL/6 mice is higher than in healthy humans, but the CV for stance width is comparable. Stride length may be determined predominantly by gait-patterning mechanisms, whereas stance width may be determined by balance-control mechanisms (Gabell et al. J Gerontol 1984, 39: 662-666). Stride length may be more variable in mice because of a greater number of gait patterns (Kale et al. Alcohol Clin Exp Res 2004, 28: 1839-1848). Gait variability may also be high in mice walking on a treadmill belt at a speed of 34 cm/s compared to mice walking overground at preferred speeds. Although pathology of PD and HD involve different portions of the basal ganglia, postural instability is common to both diseases. In patients with ALS, gait variability has been shown to be higher with well-established ALS (Hausdorff et al. J Appl Physiol 2000, 88: 2045-2053).

Without wishing to be bound by theory, it is understood that any suitable means for the measurement of gait may be used in the methods of the invention. For example, in one embodiment, the apparatus can take the form of a gait imaging system, which includes a moveable belt track upon which a subject can ambulate. In one embodiment, the imaging system includes one or more imaging devices for recording the gait of an ambulating subject on the belt track. In one embodiment, an imaging device is disposed below the belt track to record contact between at least one forelimb of the subject and the belt track. However, it is understood that one or more imaging devices could be disposed anywhere with respect to the belt track, as long as such devices are able to record the gait of a subject ambulating on the belt track. The subject can ambulate along the belt track in a substantially stationary location above the imaging device as the belt track moves, and the imaging device can record the contact by the subject.

While certain aspects of the present invention provide for the measurement of increased stride length in a subject in comparison to a suitable control to be indicative of a neurodegenerative disease in the subject, one of ordinary skill in the art will recognize that stride length can also be measured as below or equal to a suitable control value in a subject identified as having, or at risk of developing, a neurodegenerative disease, based on measurement of the state disease progression and/or other indices in the subject.

An exemplary gait measuring system that can be used in the screening methods of the invention is disclosed in U.S. Pat. No. 6,899,686 to Hampton.

Ventral Plane Videography

Ventral plane videography was recently described, and employs a high-speed digital camera to image the underside of mice walking on a transparent treadmill belt (Kale et al. Alcohol Clin Exp Res 2004, 28: 1839-1848; Hampton et al. Physiol Behav 2004, 82: 381-389). The technology generates “digital paw prints,” providing spatial and temporal indices of gait. Image capture and processing was performed in collaboration with Advanced Digital Vision (Natick, Mass.).

To measure gait dynamics, digital video images of the underside of mice were collected with a high-speed imaging device, for example at 80 frames per second, with one high-speed digital video camera from below a transparent motorized treadmill belt and stored in Audio Video Interleaved (AVI) format. Custom-developed software was used to create true color 24-bit images using Joint Photographic Experts Group (JPEG) standards. Each image represented an instant in time; when capturing at 80 fps, one frame represented 12.5 ms, for example; the paw area indicated the temporal placement of the paw relative to the treadmill belt. A mathematical representation of the color of the paws within one image, in which each of the paws was visible, was generated and used as a color reference for the entire set of images. The color images were converted to their binary matrix equivalents, and the areas (in pixels) of the approaching or retreating paws relative to the belt and camera were calculated throughout each stride. Plotting the area of each digital paw print (paw contact area) imaged sequentially in time provided a dynamic gait signal, representing the temporal record of paw placement relative to the treadmill belt. A digital mask was superimposed over the snout in all of the acquired video images of the walking mouse, based on the symmetry and direction of the animal, to prevent the snout from being misinterpreted as a paw.

The gait signals comprised a stride interval, which included the stance duration when the paw was in contact with the walking surface, plus the swing duration when the paw was not in contact with the walking surface. Stance duration was further subdivided into either braking duration (increasing paw contact area over time) or propulsion duration (decreasing paw contact area over time). Full stance was determined as the time point at which the paw contact area was maximum. The projections of the paw profile down to the surface of the treadmill belt were sometimes visible during early swing, after the paw was lifted from the belt, and prior to the next stance. Each pixel was vectorized, therefore, to improve accuracy in differentiating stance from swing.

Motor Function Measurement in MPTP- and 3NP-Treated Mice, and in SOD1 Mutant Mice

Gait in MPTP-Treated Mice

The MPTP-treated mouse model of PD has been extensively studied for its ability to injure the nigrostriatal dopaminergic system, damage neurons, and deplete the brain of dopamine (Kopin I J. Environ Health Perspect 1987, 75: 45-51; Sedelis et al. Behav Genet 2000, 30: 171-182; Jakowec et al. Comp Med 2004, 54: 497-513). Several studies have described motor function disturbances in MPTP-treated mice to relate the deficits to symptoms in humans with PD. Motor function tests in MPTP-treated mice have included grip strength (Colotla et al. Neurotoxicol Teratol 1990, 12: 405-407), the ability of the animals to balance on a rotating rod (Rozas et al. J Neurosci Methods 1998, 83: 165-175; Colotla et al. Neurotoxicol Teratol 1990, 12: 405-407), and swimming performance (Muralikrishnan et al. FASEB J 1998, 12: 905-912). MPTP significantly affects locomotor activity (Sedelis et al. Behav Genet 2000, 30: 171-182; Colotla et al. Neurotoxicol Teratol 1990, 12: 405-407; Rousselet et al. Neurobiol Dis 2003, 14: 218-228) and motor performance (Sedelis et al. Behav Genet 2000, 30: 171-182; Sedelis et al. Behav Brain Res 2001, 125: 109-125; Willis et al. Brain Res 1987, 402: 269-274; Muralikrishnan et al. FASEB J 1998, 12: 905-912), thus providing functional readouts to test potential therapies. Shortened stride length is one of the cardinal features of PD (Salarian et al. IEEE Trans Biomed Eng 2004, 51: 156-159; Nieuwboer et al. Mov Disord 2001, 16: 1066-1075; Schaafsma et al. J Neurol Sci 2003, 212: 47-53), yet reports of reduced stride length in MPTP-treated animals are sparse. Fernagut et al., using the paw-inking method, measured stride length in mice one week after acute MPTP intoxication (Fernagut et al. J Neurosci Methods 2002, 113: 123-130) and concluded that stride length was a reliable indicator of basal ganglia dysfunction. Smaller doses of MPTP (3 mg/kg) were also found to significantly reduce stride length in rats (Tsai et al. Neurosci Lett 1991, 129: 153-155). The difficulties associated with the paw-inking method and the variability in overground walking speeds in mice (Clarke et al. Physiol Behav 1999, 66: 723-729) have possibly limited reports of stride length in MPTP-treated mice. Using digital paw prints obtained by ventral plane videography, it was discovered that stride length was significantly decreased in MPTP-treated mice after 3 days of administration (i.p. 30 mg/kg/day).

Fleming et al. studied mice overexpressing wild-type human α-synuclein (ASO mice), a model of early onset familial PD (Fleming et al. J Neurosci 2004, 24: 9434-9440). The authors found that although stride length was comparable to control mice, stride frequency and stride length variability were increased in ASO mice (ibid). ASO mice did not exhibit a loss of dopaminergic neurons, but developed accumulation of α-synuclein in the nigrostriatal system and show enhanced sensitivity of nigrostriatal neurons to MPTP administration (ibid).

Gait in 3NP-Treated Mice

Fernagut et al. found no differences in stride length of forelimbs and hind limbs after a cumulative dose of 3NP (340 mg/kg) (Fernagut et al. Neuroscience 2002, 114: 1005-1017). With a cumulative dose of 560 mg/kg of 3NP, forelimb stride length was comparable to saline-treated mice, but hind limb stride length was shortened (ibid). Administration of 3NP may affect hind limb gait dynamics differently than forelimb gait dynamics via different effects on the neostriatum and the nucleus accumbens (Fernagut et al. J Neurosci Methods 2002, 113: 123-130; Cools et al. Brain Res Bull 1991, 26: 909-917). Shimano et al. showed that hind limb muscles in 3NP-treated rats became hypotonic with low voltage electromyogram activity and impaired movement (Shimano et al. Obes Res 1995, 3 Suppl 5: 779S-784S). Activation of the motor program required for the two 3NP-treated mice that braced their hind limbs against the inside walls of the running compartment while simultaneously maintaining coordinated gait of the forelimbs (Abernethy et al. Gait Posture 2002, 15: 256-265) indicated that 3NP-induced cognitive defects (Shear et al. Neuroreport 2000, 11:1833-1837) did not contribute to the gait disturbances in 3NP-treated animals.

Lin et al. reported that stride length and stance width in a knock-in mouse model of HD did not differ from wild-type mice (Lin et al. Hum Mol Genet 2001, 10:137-144). Stride length variability and stance width variability were higher, however, in the mutants (ibid). In a transgenic mouse model for HD, R6/2 mice exhibited unevenly spaced shorter strides, staggering movements, and an abnormal step sequence pattern (Carter et al. J Neurosci 1999, 19: 3248-3257). No significant abnormalities in stride length were observed in the R6/1 H) transgenic mouse (Naver et al. Neuroscience 2003, 122: 1049-1057).

Gait in SOD1 G93A Mice

Impaired performance in SOD1 G93A mice in some motor function tests has been observed at ˜8 weeks of age (Barneoud et al. Neuroreport 1997, 8:2861-2865). Others have reported motor impairments in SOD1 G93A mice at ˜11-16 weeks of age (Fischer et al. Exp Neurol 2004, 185: 232-240; Puttaparthi et al. J. Neurosci 2002, 22: 8790-8796). Increased stride length is often associated with increased amplitude of electromyogram activity and enhanced motor performance. Gurney et al. first described significantly shorter stride length in SOD1 G93A mice with severe pathological changes in the late stage of disease (Gurney et al. Science 1994, 264: 1772-1775). Puttaparthi et al. also reported significantly shorter stride length in SOD1 G93A mice at ˜24 weeks of age (Puttaparthi et al. J Neurosci 2002, 22: 8790-8796). The reported decrease in stride length at later stages could be due to muscle weakness, fatigue, and motor neuron loss.

The data of Puttaparthi et al. also indicated that stride length in SOD1 G93A mice tended to be longer at ˜16 weeks of age (ibid). Wooley et al. recently reported significantly longer stride duration in SOD1 transgenic mice compared to wild-type mice walking on a treadmill at 23 cm/s at 8 and 10 weeks of age (Wooley et al. Muscle Nerve 2005, 32: 43-50. It is noted that patients with ALS who walked overground at speeds comparable to healthy subjects also had longer stride duration (Hausdorff et al. J Appl Physiol 2000, 88: 2045-2053).

Kuo et al. identified significantly elevated intrinsic electrical excitability in cultured embryonic and neonatal mutant SOD1 G93A spinal motor neurons (Kuo et al. J Neurophysiol 2004, 91: 571-575). Dengler et al. surmised that new motor unit sprouting and resulting increases of twitch force could compensate for the loss of motor neurons in patients with early stages of ALS (Dengler et al. Muscle Nerve 1990, 13: 545-550). It was also recently reported that ALS disease progression can be monitored via measurement of motor unit number estimation (MUNE) and the neurophysiologic index (NI) in an ALS subject (de Carvalho, M., et al. Neurology 2005 May 24;64(10):1783-5).

III. Screening Assays

A number of methods of the invention relate to identifying and/or characterizing potential pharmacological agents, e.g., identifying new pharmacological agents from a collection of test substances and/or characterizing mechanisms of action and/or side effects of known pharmacological agents.

The invention provides methods (also referred to herein as “screening assays”) for identifying agents, i.e., candidate or test compounds or agents (e.g., peptides, peptidomimetics, peptoids, small molecules or other drugs) which have the effect of preventing, delaying the onset of, and/or treating ALS and/or the symptoms of ALS. Such assays typically comprise administration of a test compound to a subject (e.g., an SOD1 mutant mouse) at risk of developing ALS, predisposed to develop ALS and/or having early ALS.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

In certain embodiments, test agents of the present invention may comprise compounds present in a synthetic compound library, library of small molecules, etc. A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. In certain embodiments, a library of test agents may comprise a library, or agents drawn from a library, that is a natural product library, e.g., a library produced by a bacterial, fungal, or yeast culture.

Libraries of compounds may also be presented in solution (Biotechniques 13: 412 (1992)), or on beads (Nature 354:82 (1991), on chips (Nature 364:555 (1993), bacteria (U.S. Pat. No. 5,223,409), spores (U.S. Pat. No. 5,233,409), plasmids (PNAS USA 89:1865 (1992) or on phage (U.S. Pat. No. 5,233,409).

IV. Methods of Treatment

The present invention provides for both prophylactic and therapeutic methods of treating a subject having or at risk of (or susceptible to) a neurological disorder, e.g., ALS. “Treatment”, or “treating” as used herein, is defined as the application or administration of a therapeutic agent (e.g., a beta-blocker, e.g., propranolol) to a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a neurodegenerative disease or disorder or condition associated with such a disease or disorder (or risk of developing such a disease or disorder), by administering to the subject a therapeutic agent (e.g., a beta-blocker, e.g., propranolol). Exemplary embodiments feature methods for administration of a beta-blocker to a subject for prevention of ALS in the subject. Beta-blockers and other agents identified by the methods of the invention to prevent, reduce or delay progression of a neurological disease or disorder, e.g., ALS, in a subject may also be used therapeutically to in a subject having a neurological disease or disorder, e.g., ALS. Subjects at risk for a disease which is caused or contributed to by motor neuron degeneration can be identified by, for example, any or a combination of the diagnostic or prognostic assays involving measurement and/or observation of indices of gait dynamics and/or gait variability as described herein. Administration of a prophylactic agent can occur prior to the manifestation of symptoms characteristic of the neurodegenerative disease, such that a disease or disorder is prevented or, alternatively, delayed in its progression. A beta-blocker agent, such as propranolol, can be used in such prophylactic methods; alternatively, an appropriate prophylactic agent can be determined based on screening assays described herein. In one exemplary embodiment, prophylactic treatment of military personnel can be performed. Military personnel have an increased risk of ALS (Weisskopf, M G, et al. Neurology 2005 Jan. 11;64(1):32-7). Accordingly, providing the military with beverages or beverage kits containing an agent such as propranolol is likely to result in reducing the risk of military subjects developing neural and/or muscular symptoms of ALS and/or other neurodegenerative disorders.

2. Therapeutic Methods

Another aspect of the invention pertains to methods of modulating neurodegenerative disease progression and/or symptoms associated with a neurodegenerative disease or disorder for therapeutic purposes. Accordingly, in an exemplary embodiment, the modulatory method of the invention involves contacting a subject having a neurological disease or disorder (e.g., ALS) with an agent (e.g., a beta-blocker, e.g., propranolol) such that disease progression and/or symptom(s) associated with the disease or disorder is reduced. For the methods of the present invention, administration is performed in vivo (e.g., by administering the agent to a subject). As such, the present invention provides methods of treating an individual afflicted with a disease or disorder.

3. Pharmacogenomics

The therapeutic agents (e.g., beta-blockers, e.g., propranolol) of the invention can be administered to individuals to treat (prophylactically or therapeutically) neurodegenerative disease, disorders or symptoms associated with neurodegeneration. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a target gene polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a therapeutic agent of the present invention can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a therapeutic agent, as described herein.

Therapeutic agents can be tested in an appropriate animal model. For example, the agents as described herein that are identified and/or used to prevent and/or treat neurodegenerative disease and/or associated symptoms in a subject can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

V. Pharmaceutical Compositions

The invention pertains to uses of the above-described agents for prophylactic and/or therapeutic uses and/or treatments as described infra. Accordingly, the agents of the present invention can be incorporated into pharmaceutical compositions suitable for administration. Such compositions typically comprise the agent or compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the exemplary methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In certain embodiments, the agent(s) (e.g., beta-blocker(s), e.g., propranolol) and/or pharmaceutical compositions of the invention can be orally administered when forumulated in liquid, e.g., water, apple juice or other juice, consumed by a subject. One of skill in the art will recognize that in addition to water, a range of juices or other flavored liquids can be used to dissolve the agents of the invention (the agent(s) of the invention can also be forumulated in, e.g., a solvent or non-aqueous liquid, prior to further dissolution of the agent in an aqueous liquid for consumption by the subject). A wide range of fruit juices may promote consumption of an agent by a subject, as juices can mask flavors and/or olfactory cues associated with the agent or pharmaceutical composition that do not appeal to a subject, or may add flavors that stimulate consumption of a liquid by a subject. In certain embodiments, the present invention also contemplates the beneficial impact of administration of antioxidants in combination with the agent(s) of the invention. Accordingly, dissolving the agents of the invention in a fruit, vegetable, or other juice, e.g., a grape or berry juice, with antioxidant properties is also contemplated. Exemplary use of berry juices, and compositions derived from berry juices, for provision of antioxidant activity is described, e.g., in US Patent Application No. 20050136141.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

VI. Kits

The invention provides kits containing the constructs, or components (e.g., beta-blocker agents, antioxidant agents, and/or other neurodegenerative disease preventing, treating and/or modulating agents) necessary to make the constructs or compositions of the invention. Kits containing the pharmaceutical compositions of the invention are also provided.

Also encompassed by this invention are kits for treating, preventing and/or managing a neurodegenerative disease. Such a kit includes, for example, a beta-blocker and at least one other compound chosen from: an antioxidant, a juice (e.g., apple juice) or extract thereof, an additional beta-blocker compound, and an agent capable of preventing weight loss. Kits might further include a device, for example, for administering the compounds described herein. Additionally, kits may include instructions for administration of one or more compounds in the compositions and/or promotional materials such as, for example, marketing materials and/or any documents promoting the use of the compounds in the compositions.

In a particular embodiment, a kit for treating, preventing or managing a neurodegenerative disease featured herein includes a beta-blocker and instructions and/or promotional materials for using the compound in combination with an antioxidant compound. In another embodiment, a kit includes at least one compound selected from: a beta-blocker compound, an antioxidant, a juice (e.g., apple juice) or extract thereof, an additional agent capable of preventing, treating and/or modulating neurodegenerative disease, and an agent capable of preventing weight loss with instructions and/or promotional materials for using the compound in combination with a beta-blocker compound. Exemplary additional neurodegenerative disease preventing, treating and/or modulating agents include AM1241, ketogenic diet, ketones, colivelin, thalidomide, lenalidomide, matrix metalloproteinases, Ro 28-2653, L-carnitine, epigallocatechin gallate (EGCG, a constituent of green tea), memantine, insulin-like growth factor-1, pioglitazone, manganese porphyrin, galectin-1 and arimoclomol.

In one embodiment, kits featured herein include instructions and/or promotional materials for administration with an additional therapeutic agent based upon the functional relationship between the agents. For example, a compound having a beta-blocker may be packaged with an instructional insert which details the administration of the compound with a second compound (e.g., an antioxidant) such that they work synergistically. In other examples, a beta-blocker compound may be packaged with an instructional insert and/or promotional materials which details the administration of the compound with a second compound such that they work additively. In still other examples, a beta-blocker compound may be packaged with an instructional insert which details the administration of the compound with a second compound and further in combination with a carrier or other therapeutic agent such that their activities do not interfere with each other. It is understood that in practicing the method or using a kit of the present invention that administration encompasses administration by different individuals (e.g., the subject, physicians or other medical professionals) administering the same or different compounds.

This invention is further illustrated by the following examples which should not be construed as limiting. The contents of all references, patents and published patent applications cited throughout this application are incorporated herein by reference.

### EXAMPLES

Methods

Mice

C57BL/6 mice were studied for gait dynamics in neurodegenerative disease models, as this strain of mice has been shown to be sensitive to both MPTP and 3NP toxins (Fernagut et al. J Neurosci Methods 2002, 113: 123-130; Jakowec et al. Comp Med 2004, 54: 497-513; Schulz et al. Mol Cell Biochem 1997, 174: 193-197; Fernagut et al. Neuroscience 2002, 114: 1005-1017). Since PD, HD, and ALS share aspects of pathogenesis and pathology of motor dysfunction, gait dynamics were also studied in the SOD1 G93A transgenic mouse model of ALS (Gurney et al. Science 1994, 264: 1772-1775) to compare gait variability in mouse models of basal ganglia disease to a mouse model of motor neuron disease. Male C57BL/6J mice (7-8 weeks; ˜22 gm) were purchased from The Jackson Laboratory (Bar Harbor, Me.). Mice transgenic for the mutated human SOD1 G93A (TgN[SOD1-G93A]lGur) (SOD1 G93A) and wild-type human SOD1 (TgN[SOD1]2Gur) (wild-type controls) were purchased from The Jackson Laboratory (Bar Harbor, ME) when the mice were ˜7.5 weeks old. Animals were maintained on a 12-hour light: 12-hour dark schedule with ad libitum access to food and water. Handling and care of mice were consistent with federal guidelines and approved institutional protocols.

Experimental Groups

MPTP. A mouse model of PD was generated in the following manner. The dopaminergic neurotoxin 1-methyl-4-phenyl-1,2,3,6-tetrahydropyridine (MPTP) (Sigma-Aldrich, St. Louis, Mo.) was dissolved in saline and was administered 30 mg/kg i.p. to 7 mice every 24 hours for 3 days (MPTP-treated mice), based on previously published studies (Colotla et al. Neurotoxicol Teratol 1990, 12: 405-407; Shimoji et al. Brain Res Mol Brain Res 2005, 134:103-108). Equivolume (0.2 ml) of saline was administered i.p. to 7 control mice every 24 hours for 3 days (saline-treated mice).

3NP. A mouse model of HD was generated in the following manner. The mitochondrial toxin 3-nitropropionic acid (3NP) (Sigma-Aldrich, St. Louis, Mo.) was dissolved in saline and was administered 3 times to 6 mice: 25 mg/kg i.p. twice, separated by 12 hours (cumulative dose of 50 mg/kg), then 25 mg/kg 24 hours later (cumulative dose of 75 mg/kg) (3NP-treated mice). Equivolume (0.2 ml) of saline was administered i.p. according to the same schedule to 6 control mice. The intoxication protocol was based on published studies (Fernagut et al. Neuroscience 2002, 114: 1005-1017; Gabrielson et al. Am J Pathol 2001, 159: 1507-1520), and pilot study observations that higher doses resulted in high mortality rates or the inability of the mice to walk at all on the treadmill belt.

SOD1 G93A transgenic mice. To compare gait variability in the MPTP and 3NP mouse models of basal ganglia disease to a mouse model of motor neuron disease, gait was also examined in a mouse model of amyotrophic lateral sclerosis (ALS). Gait dynamics in SOD1 G93A mice were measured at ages ˜8 weeks (n=3), ˜10 weeks (n=3), ˜12 weeks (n=5), and ˜13 weeks (n=5), time points at which this model has been shown to exhibit motor dysfunction (Fischer et al. Exp Neurol 2004, 185: 232-240; Puttaparthi et al. J Neurosci 2002, 22: 8790-8796; Barneoud et al. Neuroreport 1997, 8:2861-2865), and compared to wild-type control mice studied at ages ˜8 weeks (n=3), ˜10 weeks (n=3), ˜12 weeks (n=6), and ˜13 weeks (n=6).

Gait Dynamics

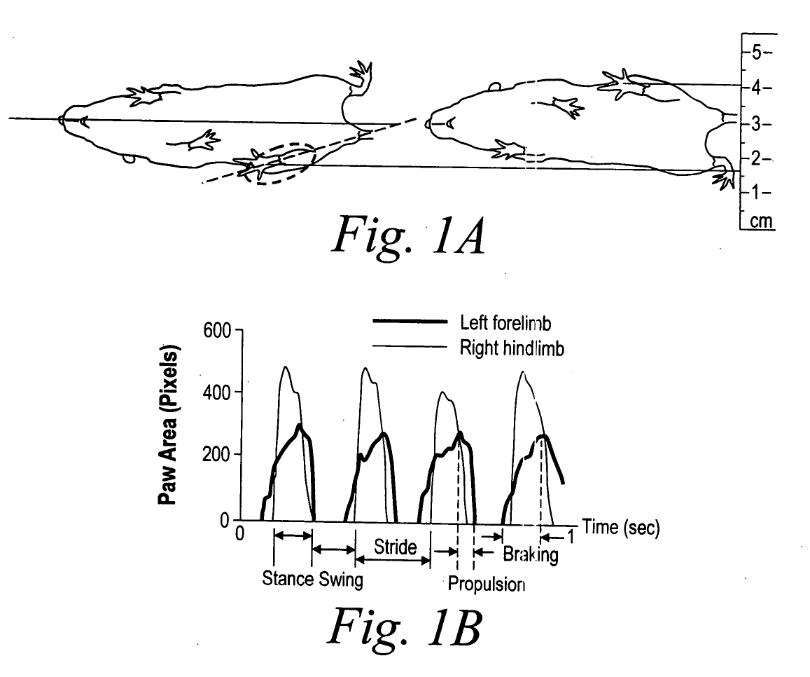

Gait dynamics were recorded using ventral plane videography, as previously described (Kale et al. Alcohol Clin Exp Res 2004, 28: 1839-1848; Hampton et al. Physiol Behav 2004, 82: 381-389). Briefly, a motor-driven treadmill with a transparent treadmill belt was built. A high-speed digital video camera was mounted below the transparent treadmill belt. An acrylic compartment, ˜5 cm wide by ˜25 cm long, the length of which was adjustable, was mounted on top of the treadmill to maintain the mouse that was walking on the treadmill belt within the view of the camera. Digital video images of the underside of mice were collected at 80 frames per second. Accordingly, each image obtained represented a 12.5 ms time interval; and the paw area of each image indicated the temporal placement of the paw relative to the treadmill belt. The color images were converted to their binary matrix equivalents, and the areas (in pixels) of the approaching or retreating paws relative to the belt and camera were calculated throughout each stride. Plotting the area of each digital paw print (paw contact area) imaged sequentially in time provided a dynamic gait signal, representing the temporal record of paw placement relative to the treadmill belt (refer to FIGS. 1A and 1B). From these digital paw print images, indices of gait and gait variability were determined. Each gait signal for each limb comprised a stride duration (stride time), which included the stance duration when the paw of a limb was in contact with the walking surface, plus the swing duration when the paw of the same limb was not in contact with the walking surface. Stance duration was further subdivided into braking duration (increasing paw contact area over time) and propulsion duration (decreasing paw contact area over time) (refer to FIG. 1B).

Stride frequency was calculated by counting the number of gait signals over time. Stride length was calculated from the equation: speed=stride frequency X stride length. To obtain stance widths and paw placement angles at full stance, ellipses were fitted to the paws, and the centers, vertices, and major axes of the ellipses were determined. Forelimb and hind limb stance widths were calculated as the perpendicular distance between the major axes of the left and right paw images during peak stance. Gait data were collected and pooled from both the left and right forelimbs, and the left and right hind limbs.

Measures of stride-to-stride variability (gait variability) for stride length, stride time, and stance width were determined as the standard deviation and the coefficient of variation (CV). The standard deviation reflected the dispersion about the average value for a parameter. CV was calculated from the equation: 100× standard deviation/mean value.

Gait was recorded ˜24 hours after each administration of saline or MPTP. Gait was recorded ˜12 hours after the 1^{st} administration, and ˜24 hours after the 2^{nd} and 3^{rd} administration of 3NP. Each mouse was allowed to explore the treadmill compartment for ˜1 minute with the motor speed set to zero, in view of previous experience with C57BU6J mice (Kale et al. Alcohol Clin Exp Res 2004, 28: 1839-1848) having indicated that such mice do not require extended acclimatization to the treadmill. The motor speed was then set to 34 cm/s and images were collected. Approximately 3 seconds of videography were collected for each walking mouse to provide more than 7 sequential strides. Only video segments in which the mice walked with a regularity index of 100% (Hamers et al. J Neurotrauma 2001; 18: 187-201) were used for image analyses. The treadmill belt was wiped clean between studies if necessary.

Statistics

Data are shown as means±SE. ANOVA was used to test for statistical differences among saline-treated, MPTP-treated, and 3NP-treated mice. When the F-score exceeded F_{critical} for α=0.05, post hoc unpaired Student's two-tailed t-tests were used to compare group means. Gait indices between forelimbs and hind limbs within the saline-treated mice were compared using Student's two-tailed t-test for paired observations. Gait indices between SOD1 G93A and wild-type control mice were compared using unpaired Student's two-tailed t-test. Differences were considered significant with P<0.05.

Stride length was significantly shorter in MPTP-treated mice (6.6±0.1 cm vs. 7.1±0.1 cm, P<0.05) and stride frequency was significantly increased (5.4±0.1 Hz vs. 5.0±0.1 Hz, P<0.05) after 3 administrations of MPTP, compared to saline-treated mice. The inability of some mice treated with 3NP to exhibit coordinated gait was due to hind limb failure while forelimb gait dynamics remained intact. Stride-to-stride variability was significantly increased in MPTP-treated and 3NP-treated mice compared to saline-treated mice. To determine if gait disturbances due to MPTP and 3NP, drugs affecting the basal ganglia, were comparable to gait disturbances associated with motor neuron diseases, we also studied gait dynamics in a mouse model of amyotrophic lateral sclerosis (ALS). Gait variability was not increased in the SOD1 G93A transgenic model of ALS compared to wild-type control mice.

### Example 1

### Gait dynamics in Saline-Treated Mice

Gait dynamics were initially examined in control (saline-treated) mice. Walking at a speed of 34 cm/s, wild type C57BU6J mice achieved ˜5 steps every second, completed one stride within ˜200 ms, and traversed ˜7 cm with each step (refer to the upper panel of FIG. 1, which depicts the ventral view of a C57BL/6J mouse walking on a transparent treadmill belt; also refer to the lower panel of FIG. 1, which displays representative gait dynamics signals for the left forelimb and right hind limb of a saline-treated mouse walking at a speed of 34 cm/s). The contributions of stance and swing durations to stride duration were ˜55% (stance/stride) and ˜45% (swing/stride), respectively. Forelimb stance width was significantly narrower than hind limb stance width (1.7±0.1 cm vs. 2.4±0.2 cm, P<0.05). The paw placement angle of the hind limbs was significantly more open than the paw placement angle of the forelimbs (13.9±1.6 vs. 2.6±0.6, P<0.05). Stride length variability of hind limbs was lower than of forelimbs (0.63±0.08 cm vs. 0.78±0.03 cm, P<0.05). Likewise, stance width variability of hind limbs was lower than of forelimbs (0.14±0.01 cm vs. 0.21±0.02 cm, P<0.05) in saline-treated mice walking on a treadmill belt at 34 cm/s.

### Example 2

### Gait Dynamics were Altered in MPTP-Treated Mice

To investigate the effect of MPTP treatment on gait, the impact of MPTP treatment on gait dynamics was assessed. Gait dynamics in MPTP-treated mice following 3 administrations of 30 mg/kg MPTP were significantly different than gait dynamics in saline-treated mice (refer to Table 1 and FIG. 2). Stride length was decreased in MPTP-treated mice compared to saline-treated mice (6.6±0.1 cm vs. 7.1±0.1 cm, P<0.05) at a walking speed of 34 cm/s. Stride frequency was increased in MPTP-treated mice. Stride duration was significantly shorter in MPTP-treated mice (194±1 ms vs. 207±2 ms, P<0.05). This was attributable to a shorter swing duration of the hind limbs (92±3 vs. 104±2 ms, P<0.05), and a shorter stance duration of the forelimbs (116±2 ms vs. 126±2 ms, P<0.05). The contributions of stance and swing to stride duration in MPTP-treated mice were not different than in saline-treated mice, despite the shorter stride duration. Forelimb stance width and hind limb stance width were comparable in MPTP-treated mice and saline-treated mice. The paw placement angles of the forelimbs and hind limbs of MPTP-treated mice were not different than in saline-treated mice. (Refer to FIG. 2, which illustrates the gait signal from the right hind limb of a MPTP-treated mouse superimposed over the gait signal from the right hind limb of a saline-treated mouse.)

Thus, it was observed that gait indices, including stride duration, stance duration, swing duration, and stride length, changed with changes in walking speed. The confounding effects of differences in walking speed on gait dynamics were eliminated by setting the motorized treadmill belt to 34 cm/s for all mice. Accordingly, since stride length was decreased in MPTP-treated mice, stride frequency was increased and stride duration was decreased in forelimbs and hind limbs of MPTP-treated mice. A decrease in stride duration can be attained by decreases in stance duration and swing duration. It was observed that the decrease in stride duration in MPTP-treated mice was attained by significantly shorter hind limb swing duration and forelimb stance duration. A reduction of the stance duration can result in a shorter time for limb muscles to be activated for stabilization (Prochazka et al. J Neurophysiol 1997, 77: 3226-3236). This likely accounted for the significant increase in stride-to-stride variability observed in MPTP-treated mice.

### Example 3

### Gait Variability was Altered in MPTP-Treated Mice

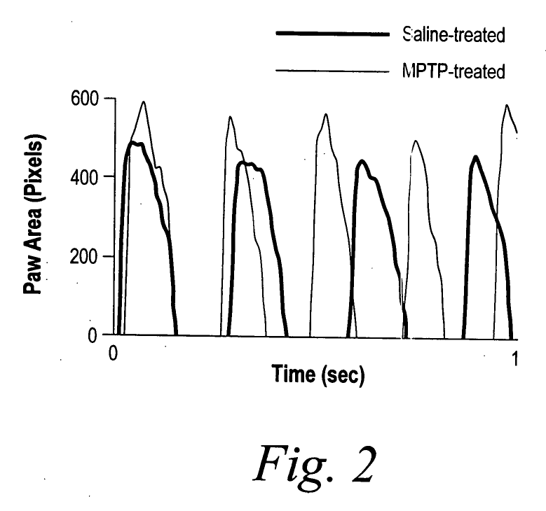

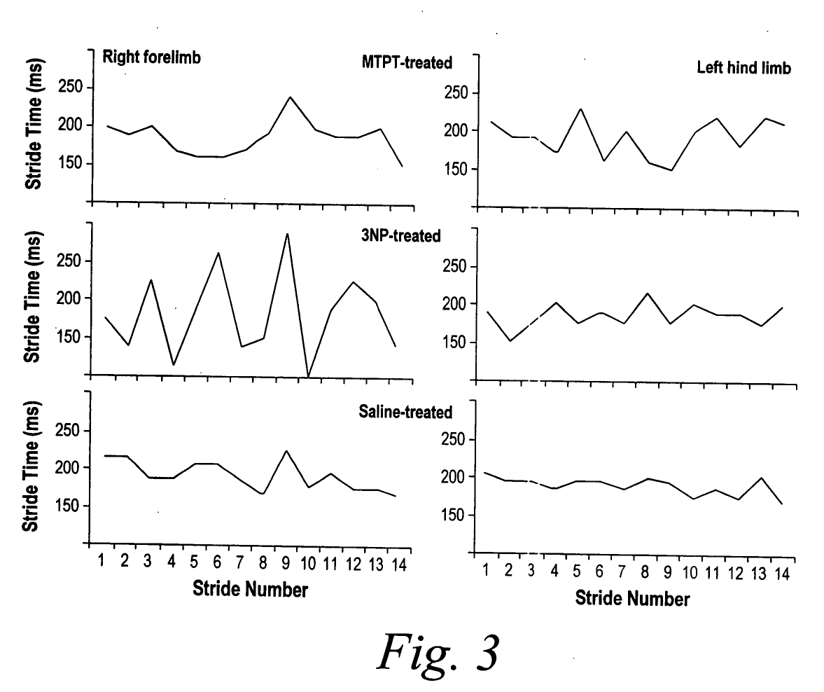

Further assessment of the effect of MPTP treatment on gait was performed by measuring the impact of MPTP treatment on gait variability. Gait variability was significantly higher in MPTP-treated mice after 3 treatments compared to saline-treated mice. Stride length variability of the forelimbs was higher in MPTP-treated than in saline-treated mice (0.91±0.04 cm vs. 0.78±0.03 cm, P<0.05). Stride length variability of the hind limbs, however, was not different in MPTP-treated mice. The coefficient of variation (CV) of forelimb stride length was significantly higher in MPTP-treated than in saline-treated mice (13.6±0.8 % vs. 11.1±0.8 %, P<0.05). The CV of hind limb stride length was somewhat higher in MPTP-treated than in saline-treated mice (10.0±1.5 % vs. 8.0±0.7 %, NS). (Refer to the top panel of FIG. 3, which shows stride time dynamics for 14 sequential strides in a MPTP-treated mouse. For comparison, stride time dynamics in a 3NP-treated mouse are illustrated in the middle panel, and in saline-treated mouse in the bottom panel of FIG. 3.)

Thus, it was observed that gait variability of the forelimbs in mice was significantly higher than gait variability of the hind limbs. This may be attributable to the role of the forelimbs in balance and navigation (Budsberg et al. Am J Vet Res 1987 48: 915-918; Cohen et al. J Morphol 1975, 146: 177-196). It was further discovered that the MPTP mouse model recapitulated the higher gait variability in patients with PD, as evidenced by a significant increase in stride length variability of the forelimbs and a significant increase in stance width variability of the forelimbs and hind limbs.

Stance width variability of the forelimbs was also significantly higher in MPTP-treated than in saline-treated mice (0.26±0.01 cm vs. 0.21±0.02 cm, P<0.05). Stance width variability of the hind limbs was higher in MFTP-treated than in saline-treated mice (0.20±0.02 cm vs. 0.14±0.01 cm, P<0.05). The CV of forelimb stance width was higher in MPTP-treated than in saline-treated mice (16.7±1.3 % vs. 12.3±1.2 %, P<0.05). The CV of hind limb stance width was higher in MPTP-treated than in saline-treated mice (9.1±1.1% vs. 5.9±0.5 %, P<0.05).

### Example 4

### Altered Gait Dynamics in 3NP-Treated Mice

To investigate the effect of 3NP treatment on gait, the impact of 3NP treatment on gait dynamics was assessed. Aggressive doses of 3NP resulted in high mortality or the inability of the mice to walk at all on the treadmill belt (data not shown). Stride length, stride frequency, stance duration, and swing duration were not affected by 3NP after the 1^{st} and 2^{nd} administrations of 25 mg/kg. The paw placement angle of the hind limbs, however, was significantly more open in 3NP-treated mice (n=6) compared to saline treated mice (16.6±1.2° vs. 12.4±1.5°, P<0.05) after the 2^{nd} administration of 3NP (cumulative dose of 50 mg/kg). Stance width variability of the forelimbs, moreover, was higher in 3NP-treated than in saline-treated mice (0.28±0.01 cm vs. 0.22±0.02 cm, P<0.05) after the 2^{nd} administration of 3NP. The CV of forelimb stance width was higher in 3NP-treated than in saline-treated mice (15.0±1.2 % vs. 11.7±0.6 %, P<0.05) after the 2^{nd} administration of 3NP. Neither stride length variability nor stance width variability of the hind limbs was affected after the 2^{nd} administration of 3NP (cumulative dose of 50 mg/kg).

After the 3^{rd} administration of 3NP (cumulative dose of 75 mg/kg), half of the 3NP-treated mice could not walk on the treadmill belt at a speed of 34 cm/s. (Observation of different effects of 3NP on gait dynamics of forelimbs and hind limbs was in accordance with previous motor behavioral assessments in 3NP-treated animals (Fernagut et al. Neuroscience 2002, 114: 1005-1017; Koutouzis et al. Brain Res 1994, 646: 242-246).) Forelimb gait indices in the three 3NP-treated mice that could walk on the treadmill belt were similar to saline-treated mice. Hind limb gait indices, however, were affected in the three 3NP-treated mice that could walk on the treadmill belt. The average hind limb stance width (2.8±0.2 cm) and paw placement angle (15.2±1.0°) in the 3NP-treated mice that could walk on the treadmill belt (n=3) was greater than in saline treated mice. The percentage of stride spent in stance was significantly greater in 3NP-treated mice than in saline-treated mice (59.4±2.3% vs. 54.3±0.9 %, P<0.05). The percentage of stance duration spent in propulsion (propulsion/stance) was greater of the hind limbs in 3NP-treated mice than in saline-treated mice (45.2±2.5 % vs. 40.2±0.9 %, P<0.05). This was at the expense of a smaller contribution of swing to stride duration (40.6±2.3 % vs. 45.7±0.9 %, P<0.05).

Stride length variability of the forelimbs, moreover, was significantly higher in the three 3NP-treated mice that could walk than in saline-treated mice (1.31±0.09 cm vs. 0.87±0.07 cm, P<0.05). Stance width variability of the forelimbs was also higher in 3NP-treated than in saline-treated mice (0.31±0.04 cm vs. 0.22±0.01 cm, P<0.05). The CV of forelimb stride length was higher in 3NP-treated than in saline-treated mice (17.9±1.6 % vs. 11.8±0.8 %, P<0.05) (refer to FIG. 3). The CV of forelimb stance width was higher in 3NP-treated than in saline-treated mice (17.3±2.4 % vs. 11.7±0.6 %, P<0.05). Hind limb stride length variability and hind limb stance width variability were not different in the 3NP-treated mice that could walk on the treadmill belt compared to saline-treated mice.

The significantly higher gait variability of the forelimbs that was observed in 3NP-treated mice likely reflects the jerky and highly variable arm movements in HD gene carriers and patients with HD (Smith et al. Nature 2000, 403: 544-549). Taken together, the presently observed increases in forelimb stride variability appeared to be more characteristic of motor control deficits in early HD than decreases in stride length.

### Example 5

### Hind Limb Gait Failure in 3NP-Treated Mice

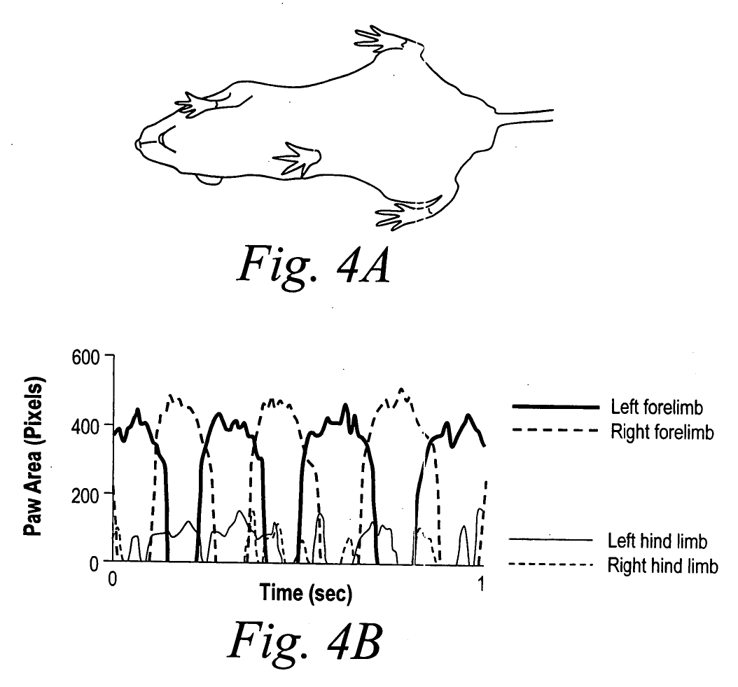

Examination of gait dynamics in 3NP-treated mice revealed hind limb gate failure in these mice. Two 3NP-treated mice that could not walk on the moving treadmill belt at a speed of 34 cm/s attempted to walk but failed to engage the hind limbs in coordinated stepping. Rather than walking, these mice braced their hind paws onto the base of the sidewalls of the running compartment (refer to FIG. 4, upper panel), avoiding the moving treadmill belt. The forelimbs of these 3NP-treated mice, however, executed coordinated stepping on the moving treadmill belt. Forelimb stride dynamics in these 3NP-treated mice did not differ significantly from saline-treated mice and the three 3NP-treated mice that were able to walk on the treadmill belt at 34 cm/s (refer to FIG. 4, lower panel). Despite the limitation of these 3NP-treated mice to only execute forelimb stepping, stride length of forelimbs was 7.1±0.1 cm, stride frequency was 5.0±0.1 Hz, and stance duration was 133±5 ms, all values similar to forelimb gait indices in saline-treated mice.

Measurements of gait dynamics in saline-treated, MPTP-treated (90 mg/kg cumulative dose), and 3NP-treated (75 mg/kg cumulative dose) mice are summarized in Table 1.

TABLE 1
Gait dynamics in saline-treated, MPTP-treated (90 mg/kg
cumulative dose), and 3NP-treated (75 mg/kg cumulative dose)
mice walking on a treadmill belt at a speed of 34 cm/s.

| | Saline | MPTP | 3NP |
|---|---|---|---|
| | (n = 7) | (n = 7) | (n = 3) |
| Stride Length (cm) | 7.1 ± 0.1 | 6.6 ± 0.1* | 7.3 ± 0.1 |
| Stride Frequency (Hz) | 5.0 ± 0.1 | 5.4 ± 0.1* | 4.9 ± 0.1 |
| Stride Duration (ms) | 207 ± 2 | 194 ± 1* | 217 ± 5 |
| % Stance Duration | 54.3 ± 0.9 | 55.9 ± 1.1 | 59.4 ± 2.3* |
| % Swing Duration | 45.7 ± 0.9 | 44.1 ± 1.1 | 40.6 ± 2.3* |
| Forelimb Stance Width (cm) | 1.7 ± 0.1 | 1.6 ± 0.1 | 1.7 ± 0.1 |
| Forelimb Paw Placement | 2.6 ± 0.6 | 2.6 ± 0.4 | 3.5 ± 1.1 |
| Angle (°) | | | |
| Hind limb Stance Width (cm) | 2.4 ± 0.2 | 2.2 ± 0.1 | 2.8 ± 0.2 |
| Hind limb Paw Placement | 13.9 ± 1.6 | 10.8 ± 1.3 | 15.2 ± 1.0 |
| Angle (°) | | | |

Means ± SE.
*P < 0.05, compared to saline-treated mice.

Thus, it was found that the 3NP mouse model likely reflects the higher gait variability that has been observed in patients with HD, as evidenced by a significant increase in forelimb stride length variability and stance width variability. It was found that gait variability of the forelimbs was highest in 3NP-treated mice, in parallel with the higher gait variability in patients with HD as compared to patients with PD (Vieregge et al. J Neural Transm 1997, 104: 237-248). As noted above, the higher forelimb stride length variability in 3NP-treated mice likely reflects the jerky movements of arms in HD patients (Smith et al. Nature 2000, 403: 544-549).

It was also noted that postural instability was characteristic of MPTP-treated and 3NP-treated mice. Increased stride length and step width variability of the hind limbs was more characteristic in the MPTP model of PD than in the 3NP-model of HD. The more open paw placement angle of the hind limbs in 3NP-treated mice was not accompanied by higher stance width variability and stride length variability. Moreover, the eventual failure of the hind limbs in 3NP-treated mice (75 mg/kg cumulative dose) to engage in coordinated stepping was not preceded by changes in hind limb gait variability (50 mg/kg cumulative dose).

### Example 6

### Gait was Altered in SOD1 G93A Transgenic Mice

Gait dynamics in the SOD1 G93A transgenic mouse model of ALS were compared with those of wild type mice in order to evaluate the impact of the SOD1 G93A mutation. Stride length was observed to be significantly greater in SOD1 G93A mice (n=5) than in wild-type mice (n=6) at ˜12 weeks and ˜13 weeks of age. At ˜12 weeks of age, stride length was significantly increased in SOD1 G93A mice compared to wild-type control mice (7.1±0.1 cm vs. 6.7±0.1 cm, P<0.05). Stride frequency was lower in SOD1 G93A mice (5.0±0.1 vs. 5.4±0.1 Hz, P<0.05), and stride duration was longer compared to wild-type control mice (210±2 vs. 197±3 ms, P<0.05) at ˜12 weeks of age. At ˜13 weeks of age, stride length remained significantly increased in SOD1 G93A mice compared to wild-type control mice (7.1±0.1 cm vs. 6.8±0.1 cm, P<0.05). Stride frequency remained lower in SOD1 G93A mice (5.0±0.1 vs. 5.3±0.1 Hz, P<0.05), and stride duration remained longer compared to wild-type control mice (209±2 vs. 198±3 ms, P<0.05) at ˜13 weeks of age. Thus, gait was found to be more “athletic” in SOD1 G93A mice at ˜12 weeks and ˜13 weeks, as compared to saline-treated mice. In view of past reports by other groups of motor impairments in ALS mice at ˜8 weeks of age to ˜16 weeks of age (Barneoud et al. Neuroreport 1997, 8:2861-2865; Fischer et al. Exp Neurol 2004, 185: 232-240; Puttaparthi et al. J Neurosci 2002, 22: 8790-8796), it was surprising to find that stride length was significantly longer in SOD1 G93A mice compared to wild-type mice at ˜12 weeks and ˜13 weeks of age. One likely explanation for the increased stride length in the presymptomatic SOD1 G93A mice that were observed walking 34 cm/s was aberrant electrical activity of the muscles involved in treadmill walking, the type of aberrant electrical activity that may be prevented or become less aberrant or more normal with or after administration of propranolol.

Gait variability was monitored in SOD1 G93A mice at ˜8 weeks, ˜10 weeks, ˜12 weeks, and ˜13 weeks of age, coinciding with the appearance of motor dysfunction reported in this model (Fischer et al. Exp Neurol 2004, 185: 232-240; Puttaparthi et al. J Neurosci 2002, 22: 8790-8796; Bameoud et al. Neuroreport 1997, 8: 2861-2865). Gait variability was not different in SOD1 G93A mice compared to wild-type control mice at age ˜8 weeks, ˜10 weeks, ˜12 weeks, and ˜13 weeks. Stride length variability of the forelimbs and hind limbs were comparable between SOD1 G93A mice and wild-type control mice at all ages studied. Stance width variability of the forelimbs and hind limbs were also comparable between SOD1 G93A and wild-type control mice at age ˜8 weeks, ˜10 weeks, ˜12 weeks, and ˜13 weeks.

Thus, no increase in gait variability was observed in transgenic SOD1 G93A mice. Neither forelimb nor hind limb stride length variability or stance width variability in SOD1 G93A mice were different than in wild-type controls at ˜12 weeks or ˜13 weeks, ages when motor function deficiencies have been observed by other groups. The present studies have demonstrated that gait variability is not increased in the early stages of motor neuron disease. Differences in gait variability among MPTP-treated, 3NP-treated, and SOD1 G93A mice likely reflect differences in neuropathology.

The present studies were the first to report stride length in subjects[mice] with ALS walking on a treadmill. As has been demonstrated herein in mice, observation of an increase in stride length can provide an early indication of ALS. Previously, there have been no studies of presymptomatic treadmill gait data from subjects who eventually developed ALS. The present observations indicate that a “more athletic” gait exists presymptomatically in subjects who eventually develop ALS. While it is known that athletes are at higher risk for developing ALS, it is also interesting to note that patients with high blood pressure are at reduced risk for developing ALS. The present invention is the first to link such a reduced risk to a protective effect of drugs prescribed for high blood pressure, such as beta-blockers.

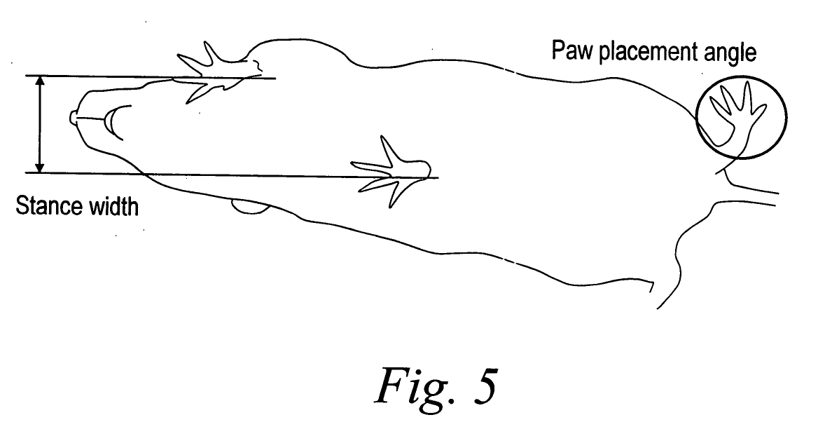

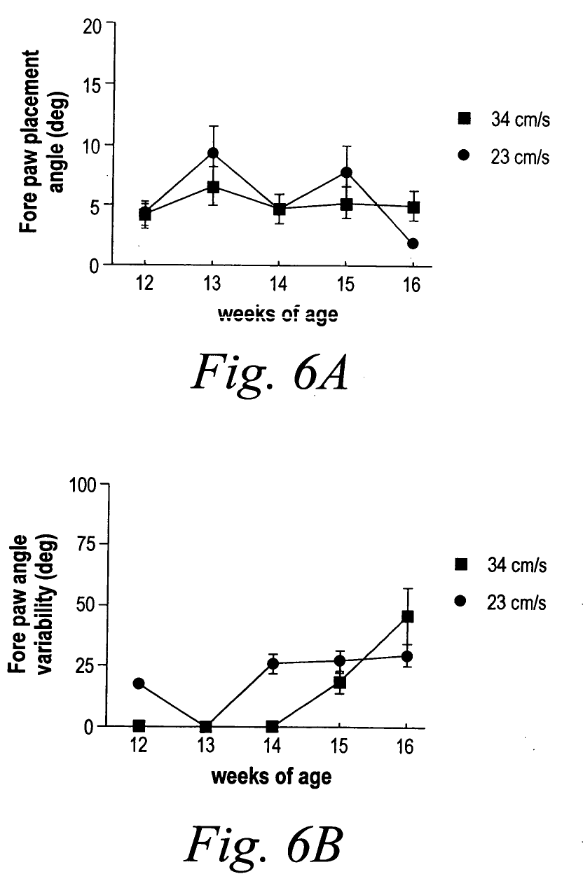

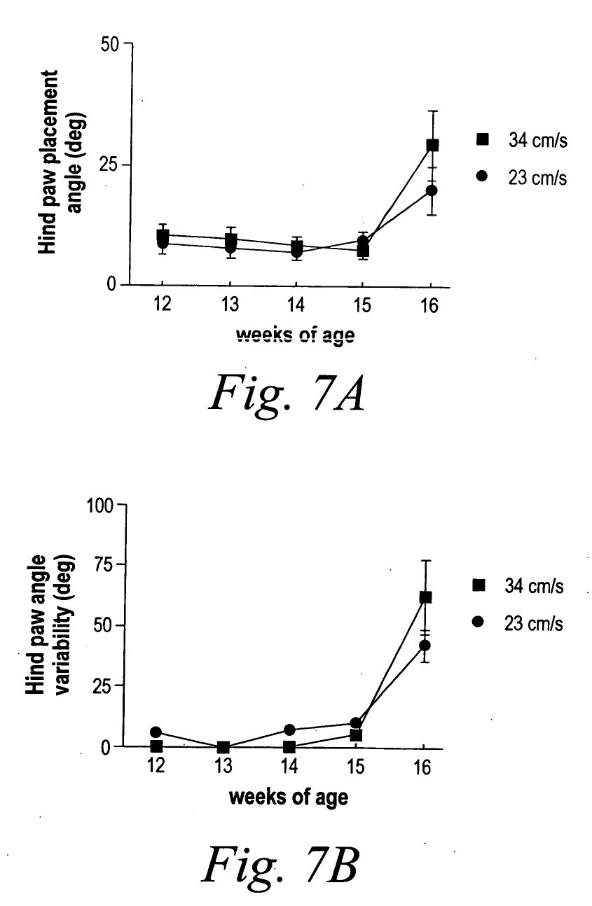

Paw placement angles were also measured for both forelimbs and hind paws of ALS mice (refer to FIG. 5 for an image that depicts measurement of both stride width and paw placement angle of a subject). Forelimb mean paw angle was observed to be relatively constant throughout stepping; however, forelimb paw angle variability rose starting at about 14 weeks of age in ALS mice (refer to FIG. 6). Hind paw placement angle of ALS mice was relatively within normal range through age about 15 weeks; however, hind paw placement angle variability was up sharply at about 15 weeks in these mice (refer to FIG. 7). Thus, it was noted that whereas many of the gait indices in ALS mice were within normal range and/or were not suggestive of disease degeneration, the index of paw placement variability, in either of the forelimbs and either of the hind limbs, was elevated in advance of obvious disease manifestation.

### Example 7

### Beta-Blockers Reduced Supranormal Gait, Delayed Onset of ALS, and Treated Symptoms of ALS in SOD1 G93A Mice

The SOD1 G93A mouse model of ALS was studied in additional experiments. As shown in the above example, gait was observed to be more “athletic” in SOD1 G93A mice prior to the development of overt symptoms of disease and neurodegeneration. Specifically, stride length, stance duration, and stride duration were prolonged in these mice compared to control mice, in advance to paralysis and death. Accordingly, it was likely that heightened sympathetic-mediated excitatory processes were reflected in the supranormal gait indices, which eventually capitulate to the processes of neurodegeneration, paralysis, and death. Blocking such sympathetic-mediated excitatory processes with a drug such as propranolol, therefore, was likely to mitigate the supranormalcy and prevent neurodegeneration, paralysis, and death.

Accordingly, five SOD1 G93A mice were treated with propranolol, the drug having been added to their drinking water beginning at six weeks of age. It was observed that propranolol added to the drinking water of SOD1 G93A mice beginning at age ˜42 days mitigated and delayed the extent of gait disturbances normally observed in these mice. Furthermore, administration of a beta-blocker was observed to modulate an ALS-associated aspect of gait. Specifically, it was observed that whereas ALS mice receiving untreated tap water exhibit longer strides, stride length was more normal in SOD1 G93A mice administered the beta-adrenergic blocking drug propranolol.

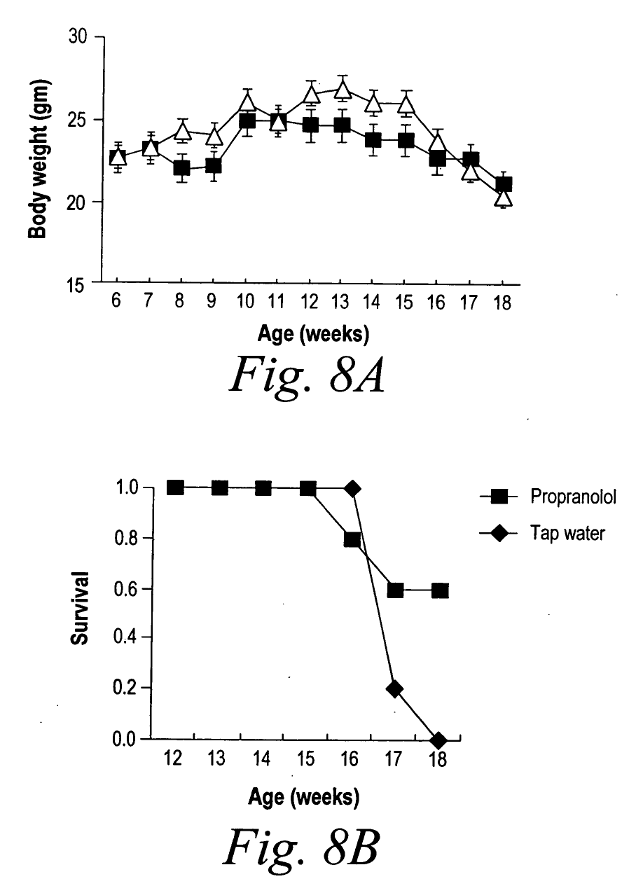

It was also observed that whereas only one out of five ALS mice receiving untreated tap water could successfully walk (i.e. engage all limbs for coordinated stepping) on a moving treadmill belt at ˜16 weeks of age, three out of five of the SOD1 G93A mice administered the beta-adrenergic blocking drug propranolol could successfully walk on a moving treadmill belt at ˜16 weeks of age. It was additionally observed that whereas only one tap-water-treated ALS mouse survived at ˜age 17 weeks, 3 propranolol-treated mice survived at ˜age 17 weeks (refer to FIG. 8). Thus, administration of a beta-blocker to SOD1 G93A ALS mice was observed to delay both onset and progression of ALS. As also shown in FIG. 8, a reduction in body weight was noted in propranolol-treated mice relative to tap water-treated control mice. Accordingly, administration of a second compound capable of preventing weight loss, in addition to administration of a beta-blocker or other prophylactic or therapeutic agent of the invention, is likely to further prevent, delay and/or mitigate the symptoms of ALS.

It was additionally observed that paw placement angle variability of ALS mice became significantly higher as the disease progressed; however, the increase in paw placement angle in propranolol-treated mice was blunted or prevented. Thus, a beneficial effect of propranolol treatment was observed in an additional phenotype associated with ALS progression in SOD1 mice.

Stance width was also observed to decrease by more than 10% in SOD1 G93A mice, in both the forelimbs and hind limbs (upper limbs and lower limbs), between age 12 and 13 weeks.

It was observed that adding apple juice to the tap water in which propranolol was dissolved ensured that the animals would consume the propranolol-laced beverage. In fact, animals consumed two to three times the normal amount of liquid if it contained the apple juice. Thus, for a short period of time during administration of propranolol, the amount of drug administered to the animals exceeded targeted levels of administration. Thus, propranolol dosing was increased by the presence of apple juice in the propranolol-laced beverage.

In addition to the observed benefit of propranolol in ALS mice, it is possible that apple juice can prevent, delay, or mitigate the symptoms of ALS. However, in these experiments, the ALS animals that were not given propranolol were exposed to the same amount of apple juice; and these animals developed symptoms and died according to a timeline that is known and accepted for ALS mice. It is therefore unlikely that apple juice per se was protective. However, it was possible that additional benefits of fruit juices, such as anti-oxidant properties, were additive to the benefits conferred by the main drug, e.g., propranolol.

In addition, it was found that paw placement angle variability increases steadily from age 12 weeks through age 18 weeks in the SOD1 G93A mouse model; the increase was prevented or diminished in the propranolol-treated SOD1 G93A mice. Whereas the paw placement angle per se did not change dramatically as the animal aged, the variability in the paw placement angle did increase as the animal aged from 12 weeks to 14 weeks. Accordingly, this metric, paw [foot] placement angle variability, can be used as a predictor of ALS (and other neurodegenerative conditions) in subjects who are prone to develop the disease, or subjects (e.g., people) with the disease at an early stage.

The distinct characteristics of gait and gait variability observed in the MPTP model of Parkinson's disease (PD), the 3NP model of Huntington's disease (HD) and the SOD1 G93A model of amyotrophic lateral sclerosis (ALS) likely reflect impairment of specific MPTP-, 3NP- and SOD1-affected neural pathways, respectively.

### Example 8

### Determination of Additional Protective or Therapeutic Effects of Beta-Blockers and Metabolites Thereof

Identification of additional protective or therapeutic effects of beta-blockers and metabolites thereof is achieved in the following manner. An SOD1 G93A mouse is administered propranolol in numerous regimens of the drug commencing prior to six weeks of age. Administration of the drug sooner, and even in utero via administration of the drug to the mother of a subject, can also be performed to provide comparable or improved efficacy. The drug is administered to different subject groups, some commencing administration in utero, others commencing in neonates, others commencing at 1 week of age, etc. The protective effect of early administration of the drug on ALS onset and/or progression is monitored via assessment of gait dynamics of the test subject groups and is compared to an appropriate control.

Similar studies are also performed to measure any beneficial impact of suspending or terminating administration of the drug prior to 17 weeks of age.

The preceding studies are also performed to assess the effect of 4-HO-propranolol (4HOP) administration on subjects. 4HOP, a major metabolite of propranolol, has antioxidant properties. Measurement of a beneficial effect upon subjects (due to both antisympathetic and antioxidant properties) is performed.

### Example 9

### Assessment of Gait Dynamics in Human Subjects

Assessment of gait dynamics can also be performed in a human subject. Neurodegenerative disease, or a risk of developing a neurodegenerative disease (e.g., ALS, or a predisposition to ALS) is tested in a subject via observation of gait dynamics in a subject crawling on his hands and knees on a walking surface. Distances between hands and between knees are measured and used to compare these metrics to “normal” values to determine whether a subject's measured values are deemed abnormal (e.g., supranormal). Measurement of abnormal values in this test indicates a neurodegenerative disease condition, e.g., ALS. The test is also performed using measurement of angles made by the hands and by the knees, relative to the centerline of the body of the subject, measured for each stride, in a predictive/diagnostic manner. Alternatively, a combination of the preceding indices (limb placement variability AND stance width) is analyzed in a predictive/diagnostic manner. Walking on hands and knees (crawling) is more like quadrapedal gait than is walking on the feet. However, assessment of crawling can provide metrics indicative of ALS not otherwise seen. Assessment of gait dynamics can also be performed in a human subject walking on a moving treadmill belt. Distances between feet, and angles that the feet make with the center line of the walking surface and/or the direction of the line of walking, can be made. Quantitative metrics of arm motion can also be used. As was observed in mice with ALS, the variability of the foot placement angle, and/or an increased stride length or increased arm swing can be indicative of ALS.

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents, patent publications and non-patent references cited in this disclosure are incorporated by reference in their entirety. The citation of any references herein is not an admission that such references are prior art to the present invention.

Unless otherwise indicated, all numbers expressing quantities of ingredients, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained by the present invention. Unless otherwise indicated, the term “at least” preceding a series of elements is to be understood to refer to every element in the series.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating or preventing early amyotrophic lateral sclerosis (ALS) in a subject in need thereof comprising administering a beta-adrenergic blocking agent (beta-blocker) to the subject, such that early ALS is treated or prevented.

2. The method of claim 1, wherein the subject has an increased stride length in comparison to a standardized average length stride.

3. The method of claim 2, wherein stride length is measured for a subject walking on a surface selected from the group consisting of a treadmill belt and the ground.

4. The method of claim 1, wherein the beta-blocker is selected from the group consisting of acebutolol, atenolol, betaxolol, bisoprolol, carteolol, celeprolol, labetalol, metoprolol, nadolol, nebivolol, oxprenolol, penbutolol, pindolol, propranolol, sotalol, esmolol, carvedilol, timolol, bopindolol, medroxalol, bucindolol, levobunolol, metipranolol, celiprolol and propafenone.

5. The method of claim 4, wherein the beta-blocker is administered to the subject via a route selected from the group consisting of parenterally, intravenously, intradermally, subcutaneously, intraperitoneally, intramuscularly, orally, transdermally and transmucosally.

6. The method of claim 1, further comprising administering a compound capable of preventing weight loss to the subject.

7. The method of claim 1, further comprising administering an antioxidant to the subject.

8. The method of claim 7, wherein the antioxidant is an isolated 4-HO-propranolol (4HOP).

9. A method for preventing the onset of ALS symptoms in a subject having early ALS comprising administering an agent which reduces at least one characteristic selected from the group consisting of excitability of a motor neuron, motor performance, and muscle strength.

10. The method of claim 9, wherein the agent is a beta-blocker.

11. A method for preventing or delaying the onset of symptoms of amyotrophic lateral sclerosis (ALS) in a subject comprising administering an effective amount of propranolol to the subject, such that early ALS is prevented.

12. The method of claim 11, wherein the subject is a member of the military.

13. The method of claim 11, wherein the propranolol is administered in combination with or formulated in apple juice.

14. The method of claim 11, wherein the propranolol is administered in combination with or formulated in a sports drink.

15. The method of claim 11, wherein the propranolol is administered in combination with or formulated in a military diet.

16. A method for diagnosing early amyotrophic lateral sclerosis (ALS) in a subject comprising
a) measuring a stride length of the subject; and
b) determining whether the stride length of the subject is increased relative to a standardized average length stride,
wherein an increase in stride length indicates ALS in the subject.

17. The method of claim 16, wherein stride length of the subject is measured on a treadmill.

18. The method of claim 16, wherein the subject has not been previously diagnosed with ALS and displays none of the neurodegenerative characteristics associated with ALS.

19. A method for identifying an agent which treats or reduces the advancement, severity or effects of ALS comprising:
a) administering the agent to an experimental vertebrate predisposed to have ALS or showing ALS symptoms;
b) measuring a stride length of said vertebrate; and
c) determining whether the stride length of the subject is decreased in comparison to a control vertebrate that has not been administered said agent,
wherein a decrease in stride length of the experimental vertebrate indicates the agent treats or reduces the advancement, severity or effects of ALS.

20. The method of claim 19, wherein the experimental vertebrate is a rodent.

21. The method of claim 20, wherein the rodent is a mouse.

22. The method of claim 21, wherein the mouse is an SOD1 mouse.

23. The method of claim 19, wherein stride length is measured by placing the experimental vertebrate on a treadmill.

24. The method of claim 19, wherein stride length is measured using ventral plane videography.

25. A pharmaceutical composition comprising the identified agent of claim 19, and a pharmaceutically acceptable carrier.

26. A method for identifying an agent which treats or reduces the advancement, severity or effects of a neurodegenerative disease comprising
a) administering the agent to an experimental vertebrate predisposed to having the neurodegenerative disease or showing signs of the neurodegenerative disease;
b) measuring a foot placement angle variability of said vertebrate; and
c) determining whether the foot placement angle variability of the vertebrate is decreased in comparison to a control vertebrate that has not been administered said agent,
wherein a decrease in the foot placement angle variability of the experimental vertebrate indicates the agent treats or reduces the advancement, severity or effects of the neurodegenerative disease.

27. The method of claim 26, wherein the neurodegenerative disease is ALS.

28. A method for testing a subject to determine whether the subject has or is at risk of developing a neurodegenerative disease comprising
a) having said subject move on said subject's forelimbs and hindlimbs on a surface;
b) measuring the distance between the subject's forelimbs and hindlimbs; and
c) comparing the measured distance to control distance,
wherein an increased difference in the measured distance compared to the control distance indicates that said subject may have or be at risk of developing a neurodegenerative disease.

29. A method for treating or preventing a neurodegenerative disease in a subject identified using the method of claim 28 comprising administering propranolol to said subject.

30. A method for testing a subject to determine whether the subject has or is at risk of developing a neurodegenerative disease comprising
a) having said subject move on said subject's limbs on a surface;
b) measuring the angles made by said subject's limbs relative to the centerline of said subject's body; and
c) comparing the measured angles to control angles,
wherein an increased difference in the measured angle compared to the control angle indicates that said subject may have or be at risk of developing a neurodegenerative disease.

31. A kit for treating ALS in a subject comprising a beta-blocker and instructions for use.

32. The kit of claim 31, further comprising an antioxidant.
